(19) United States
(12) Patent Application Publication
(10) Pub. No.: US 2023/0165205 A1
(43) Pub. Date: Jun. 1, 2023

(54) METHODS FOR INDUCTION OF ENDOGENOUS TANDEM DUPLICATION EVENTS

(71) Applicant: Academisch Ziekenhuis Leiden (h.o.d.n. LUMC), Leiden (NL)

(72) Inventors: Marcel TIJSTERMAN, Leiden (NL); Robin VAN SCHENDEL, Leiden (NL)

(21) Appl. No.: 17/919,138

(22) PCT Filed: Apr. 12, 2021
(86) PCT No.: PCT/NL2021/050237

§ 371 Date: Oct. 14, 2022

(30) Foreign Application Priority Data
Apr. 14, 2020 (NL) 2025344
Nov. 23, 2020 (NL) 2026955

(51) Int. Cl. A01H 1/06; C07K 14/415

(52) U.S. Cl. CPC A01H 1/06; C07K 14/415

(57) ABSTRACT

The present invention provides methods of deliberately increasing a rare endogenous genome modification called tandem duplication events in the cells of an organism. The invention also provides methods for identifying and/or selecting a cell with a trait of interest that is the result of such tandem duplication events. Methods for screening a population of cells and identifying and/or selecting a cell with a desired trait are also provided herein. A population of plant cells, plant parts or plants obtained by the methods described herein are also provided.

The present invention provides methods of deliberately increasing a rare endogenous genome modification called tandem duplication events in the cells of an organism. The invention also provides methods for identifying and/or selecting a cell with a trait of interest that is the result of such tandem duplication events. Methods for screening a population of cells and identifying and/or selecting a cell with a desired trait are also provided herein. A population of plant cells, plant parts or plants obtained by the methods described herein are also provided.

### BACKGROUND

Tandem duplication (TD) events occur naturally, but extremely rarely within DNA, when a DNA sequence is duplicated and positioned immediately adjacent to the DNA that acted as its template. TDs have been causally linked to phenotypic alterations of cells and organisms and are key drivers of evolution.

TDs are a prominent natural source of genetic diversity and also very advantageous for the development of novel traits because gene duplications allow the duplicated copy to obtain new molecular functions while the original copy prevents a selective penalty. Gene duplications may further increase the expression of a certain gene and thereby perturb the normal homeostasis of cells. The latter event could have immediate and also selective advantages (e.g. duplication of growth factors may result in increased growth).

Although TD formation has been observed in species from all kingdoms and can provide species with a rich source of genomic diversity, the mechanism by which TDs form is currently unknown (Wang et al., 2015). In addition, the rate with which TDs arise naturally is uniformly very low across species. This prevents TDs from being used as drivers of genetic change by molecular biologists or plant breeders.

Present plant breeding technology either uses i) random mutagenesis by chemical exposure or radiation (for example), which induces almost exclusively loss of function alleles, which have limited benefits with respect to trait improvement, ii) elaborate crossing schemes to employ/combine naturally occurring trait differences, or iii) transgenesis, but only if there is tremendous knowledge about the biology associated with the gene.

There is a need to develop improved technologies for trait development.

### BRIEF SUMMARY OF THE DISCLOSURE

The inventors have discovered that the gene TONSOKU is implicated in preventing tandem duplication events from occurring within genomes. Gene deletion experiments have revealed that the protein encoded by TONSOKU prevents or suppresses the random formation of genomic duplications in the nematode Caenorhabditis elegans and the plant Arabidopsis thaliana. Therefore, the function of this gene is evolutionarily conserved in animals and plants.

The inventors have found that nematodes and plants with mutated TONSOKU accumulate tandem duplications in their genome at a significantly higher rate than their respective wild-type organisms. Such tandem duplication events are not deleterious and once homozygous the net effect is a random doubling of the expression for a number of closely positioned genes.

The inventors have utilized the reduction in TONSOKU protein expression to increase the rate of tandem duplication events within plant genomes, thereby increasing genetic variation. The methods described herein therefore provide an entirely novel way of changing the genetic content (or homeostasis) of an organism (e.g. a plant) by addition instead of reduction that can be used for trait development.

In one aspect, there is provided a method of increasing endogenous genome modification in a plant cell, wherein the method comprises: reducing or abolishing the expression of at least one TONSOKU nucleic acid sequence and/or reducing or abolishing the level of a TONSOKU polypeptide and/or reducing or abolishing an activity of a TONSOKU polypeptide in the plant cell.

Suitably, the method may increase endogenous insertions within the genome of the plant cell.

Suitably, the methods described herein may result in at least one tandem duplication event occurring within the genome of the plant cell. Alternatively, the methods described herein may result in at least two tandem duplication events occurring within the genome of the plant cell, wherein the at least two tandem duplication events occur at different locations within the genome. As a further alternative, the method described herein may result in at least three tandem duplication events occurring within the genome of the plant cell, wherein the at least three tandem duplication events occur at different locations within the genome.

Suitably, each tandem duplication event as described herein can occur at a random location within the genome of the plant cell.

Suitably, a unit sequence that is repeated by a tandem duplication event can be 50-500 kilobases in size.

Suitably, the methods described herein may comprise introducing at least one mutation into:
(i) the at least one TONSOKU gene;
(ii) an upstream promoter of the at least one TONSOKU gene; or
(iii) a regulatory element of the at least one TONSOKU gene.

Suitably, the mutation could be a loss of function mutation. Suitably, the mutation can be an insertion, deletion or substitution.

Suitably, the mutation can be introduced using a targeted genome modification technique. Suitably, the targeted genome modification technique may be selected from CRISPR/Cas9, ZFNs, TALENs or meganucleases.

Suitably, the mutation can be introduced using mutagenesis. Suitably, the mutagenesis could be selected from: EMS, TILLING, transposon or T-DNA insertion.

Suitably, the plant cell may be homozygous for the mutation.

Suitably, the methods described herein can comprise using RNA interference to reduce or abolish the expression of the at least one TONSOKU nucleic acid sequence in the plant cell.

Suitably, the TONSOKU nucleic acid sequence can comprise or consist of SEQ ID NO: 3 or 4.

Suitably, the method may comprise use of an inhibitor to reduce or abolish an activity of the TONSOKU polypeptide in the plant cell.

Suitably, the TONSOKU polypeptide may comprise or consist of SEQ ID NO: 1.

Suitably, the increase in endogenous genome modification in the plant cell can be relative to a control plant cell or a wild-type plant cell.

Suitably, the plant cell could be in a plant tissue, such as pollen, ovules, leaves, embryos, roots, root tips, anthers, flowers, fruits, stems shoots or seeds.

Suitably, the plant cell as described herein may be in a plant part, such as pollen, ovules, leaves, embryos, roots, root tips, anthers, flowers, fruits, stems, shoots, scions, rootstocks, seeds, protoplasts or calli.

Suitably, the plant cell could be in a plant. Suitably, the plant can be selected from: cotton, cantaloupe, radicchio, papaya, plum, peanut, oilseed rape, canola, sunflower, safflower, olive, sesame, hazelnut, almond, avocado, bay, pumpkin/squash, linseed, soya, pistachio, borage, maize, wheat, rye, oats, sorghum and millet, triticale, rice, barley, cassava, potato, sugarbeet, egg plant, alfalfa, perennial grasses, forage plants, oil palm, vegetables (brassicas, root vegetables, tuber vegetables, pod vegetables, fruiting vegetables, onion vegetables, leafy vegetables and stem vegetable), buckwheat, Jerusalem artichoke, broad bean, vetches, lentil, dwarf bean, lupin, clover, lucerne, tobacco, tomato, ornamental plants and marijuana.

Suitably, the methods described herein may further comprise the step of: (ii) growing the plant to seed. Suitably, the methods described herein may further comprise the step of (iii) growing the seed(s) obtained in step (ii). Suitably, the method can further comprise repeating steps (ii) and (iii) as described herein.

Also provided herein is a method for identifying and/or selecting a plant cell with a trait of interest, the method comprising:
(i) reducing or abolishing the expression of at least one TONSOKU nucleic acid sequence and/or reducing or abolishing the level of a TONSOKU polypeptide and/or reducing or abolishing an activity of a TONSOKU polypeptide in the plant cell;
(ii) selecting at least one plant cell with a trait of interest; and optionally
(iii) genotyping the plant cell obtained in step (ii).

Suitably, the methods as described herein may further comprise growing the plant cell obtained in step (i). Suitably, the methods as described herein may further comprise growing the plant cell obtained in step (i) into a plant. Suitably, the methods as described herein may further comprise growing the plant to seed to obtain progeny of the plant.

Suitably, the selection of at least one plant cell with a trait of interest can be determined by:
(i) inspecting morphological features of the at least one plant cell;
(ii) genotyping the at least one plant cell;
(iii) transcriptomic analysis of the at least one plant cell;
(iv) metabolomic analysis of the at least one plant cell; or
(v) assessing the behaviour of the at least one plant cell in a phenotypic assay.

Further provided herein, is a method for screening a population of plant cells and identifying and/or selecting a plant cell with a trait of interest, wherein the method comprises:
(i) reducing or abolishing the expression of at least one TONSOKU nucleic acid sequence and/or reducing or abolishing the level of a TONSOKU polypeptide and/or reducing or abolishing an activity of a TONSOKU polypeptide in the plant cell;
(ii) selecting at least one plant cell with a trait of interest; and optionally
(iii) genotyping the plant cell obtained in step (ii).

Suitably, the methods may further comprise growing the plant cells obtained in step (i) to form a population of plant cells. Suitably, the methods described herein may further comprise screening the population of plant cells obtained in step (i) for reduced expression of at least one TONSOKU nucleic acid sequence or a reduced level of a TONSOKU polypeptide or reduced activity of a TONSOKU polypeptide in the plant cell prior to step (ii) and (iii).

Suitably, the trait of interest can be selected from: insect resistance, disease resistance, herbicide tolerance, male sterility, abiotic stress tolerance, altered phosphorus utilisation, altered antioxidants, altered fatty acids, altered essential amino acids, altered carbohydrates, altered sequences involved in site-specific recombination, altered development, or altered morphology (such as size and pigmentation).

Also provided herein is a population of plant cells, plant parts or plants obtained by the methods as described herein.

In another aspect, described herein is the use of a plant or plant cell having reduced or abolished expression of at least one TONSOKU nucleic acid sequence and/or a reduced or abolished level of a TONSOKU polypeptide and/or reduced or abolished activity of a TONSOKU polypeptide in the plant cell for trait development, for example in the context of plant breeding.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, N Y (1994); and Hale and Marham, The Harper Collins Dictionary of Biology, Harper Perennial, N.Y. (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, polynucleotides are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Various aspects of the invention are described in further detail below.

### BRIEF DESCRIPTION OF THE DRAWINGS

Embodiments of the invention are further described hereinafter with reference to the accompanying drawings, in which:

FIG. 1 shows that large tandem duplications arise in the genomes of species with a deficiency in the gene TONSOKU/tnsl-1.

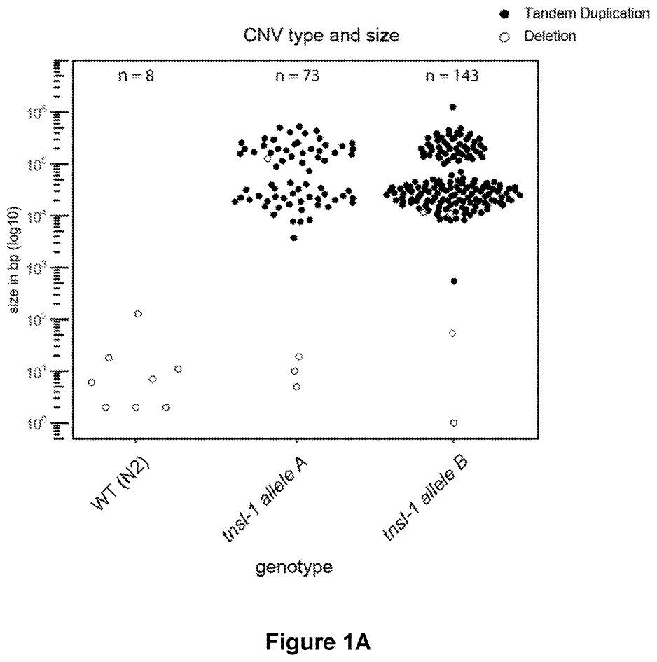

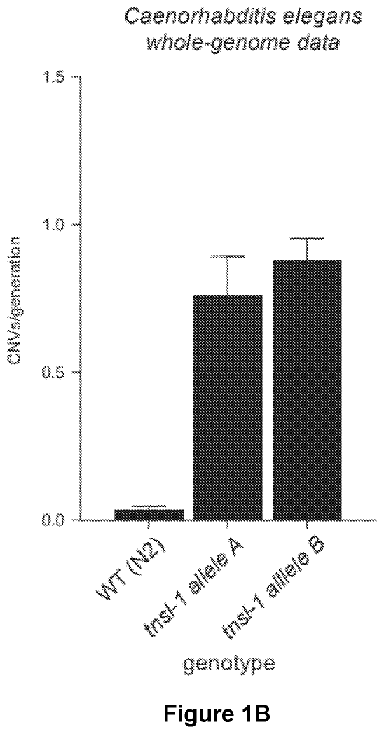

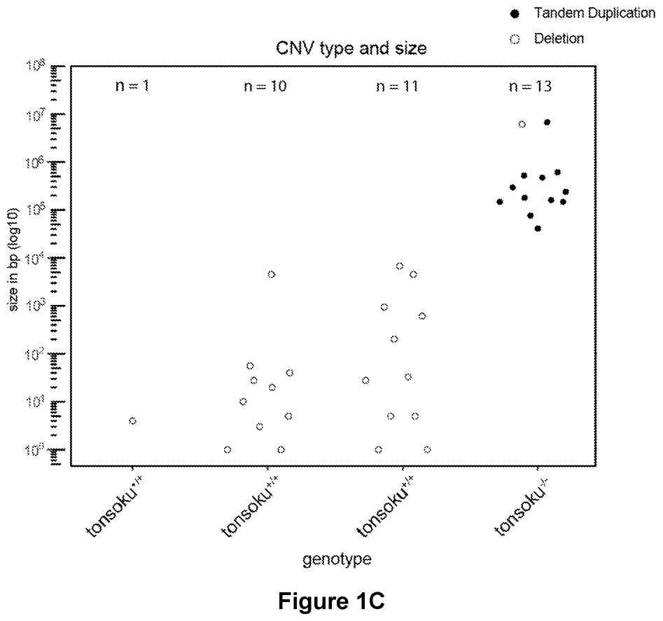

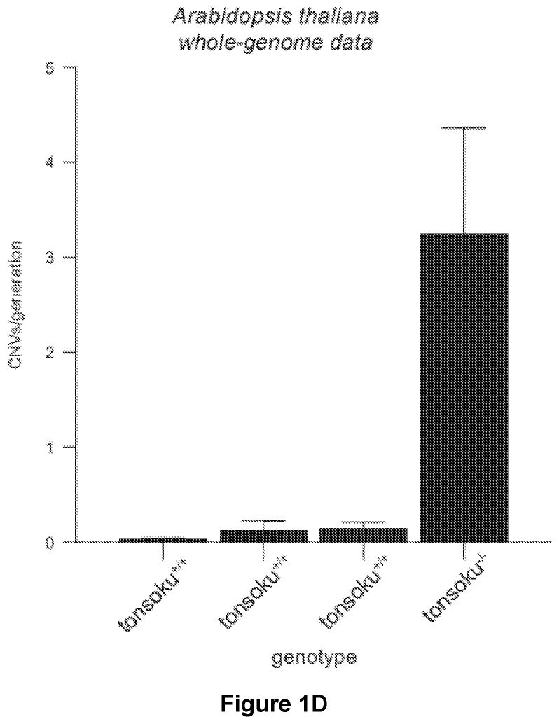

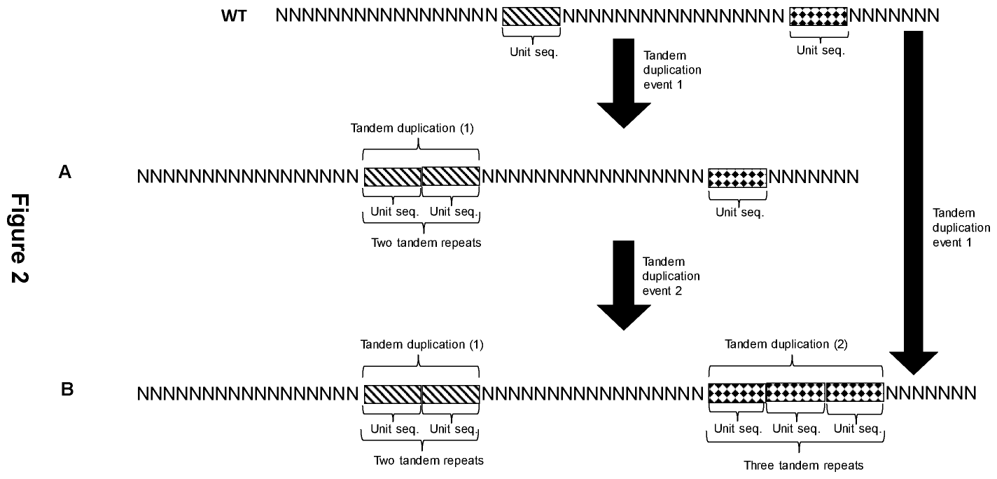

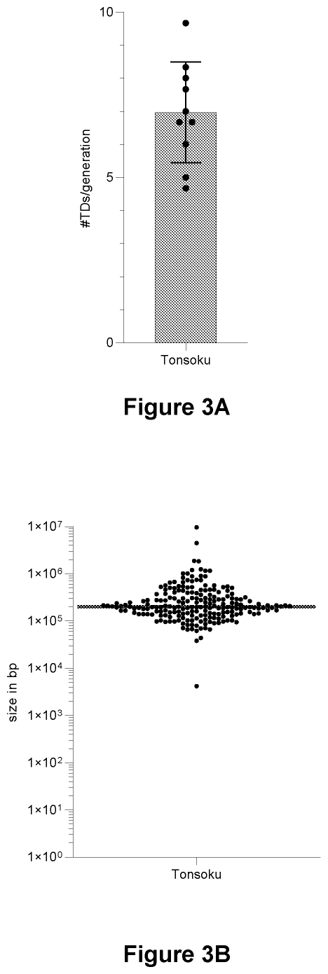

1A) Unique genome alterations found in Caenorhabditis elegans proficient (WT(N2)) and deficient for tnsl-1. Animals were grown for 150-240 generations. Tnsl-1 proficient animals did not acquire any tandem duplications after 240 generations, while two strains with different mutations in tnsl-1 (allele A and B) accumulate numerous tandem duplications during normal growth conditions.

1B) Quantification of the number of copy-number alterations (also known as copy-number variants, or CNVs) per animal generation for the indicated genotypes. For each genotype, at least three individual populations were clonally propagated for 25-60 generations. Bars represent the average CNVs/generation, error bars depict s.e.m.

1C) Unique genome alterations found in the plant Arabidopsis thaliana that are either proficient or deficient for the gene TONSOKU. Each TONSOKU proficient sample contains the genomic data of ˜18-20 plants that were grown for 5 generations: TONSOKU proficient animals did not acquire any tandem duplications in >270 generations. The TONSOKU deficient sample contains the genomic data of 4 plants that are the progeny of one homozygous parental plant. Here, 12 tandem duplication events were observed. The TONSOKU proficient lines are SALK_014731, SALK_031862 and SALK_016627. The TONSOKU deficient line is SAIL_525_A01.

1D) Quantification of the number of CNVs/generation for TONSOKU proficient and deficient plants (CNVs include TDs as well as deletions and insertions). Bars show average CNVs/generation, error bars depict s.e.m.

FIG. 2 shows a diagrammatic representation of the meaning of a unit sequence, tandem repeat and tandem duplication, and tandem duplication event(s) as used herein. 2A) shows a genome with one tandem duplication. 2B) shows a genome with two tandem duplications.

FIG. 3 shows tandem-duplication formation in Arabidopsis thaliana with a homozygous mutation in TONSOKU. A) frequency of de novo tandem duplications (TDs) per generation is shown in a bar graph. Each dot represents the frequency of tandem duplications in a single plant grown for three generations. B) A scatter plot of all de novo tandem duplications detected in 10 sublines grown for three generations. The y-axis shows the size in bp on a log-10 scale. Line represents the median tandem duplication size (199,589 bp).

### DETAILED DESCRIPTION

The inventors have surprisingly discovered that reduction of TONSOKU at either the protein or genomic level increases endogenous genome modification in a cell. This discovery is conserved in animals and plants. The invention therefore has broad utility in a variety of animal and plant systems.

The term “TONSOKU” is used herein to refer to a nucleic acid sequence of a TONSOKU gene. This gene is also referred to as “MGOUN3” and “BRUSHY1” in the literature (Guyomarc'h et al., 2006; Ohno et al., 2011). The term “TONSOKU” as used herein therefore encompasses genes referred to as “TONSOKU”, “MGOUN3” or “BRUSHY1” in the literature. Moreover, the definition encompasses any nucleic acid encoding a TONSOKU protein.

The TONSOKU gene sequence is well known by a person of skill in the art. By way of example only, the TONSOKU gene of A. thaliana has a sequence of SEQ ID NO: 3, and a promoter sequence comprising SEQ ID NO: 2. SEQ ID NO: 3 is therefore an example of an “endogenous TONSOKU gene” or “wildtype TONSOKU gene”. Similarly, SEQ ID NO: 2 is an example of an “endogenous TONSOKU promoter” or “wildtype TONSOKU promoter” herein. Other TONSOKU gene sequences found in plants are readily identifiable to a person of skill in the art. For the avoidance of doubt, the term TONSOKU therefore encompasses the sequence of SEQ ID NO:3 (optionally together with a promoter sequence comprising SEQ ID NO:2) and plant homologues thereof.

Homologues of the plant gene are also known in animals, such as “TONSL” which is also known as “NFKBIL2” (O'Donnell et al., 2010). Such homologues are readily identifiable to a person of skill in the art. The invention is therefore not limited to TONSOKU, but may also apply to non-plant homologues thereof, such as those found in animals.

As used herein, the words “nucleic acid”, “nucleic acid sequence”, “nucleotide”, “nucleic acid molecule” or “polynucleotide” include DNA molecules (e.g., cDNA or genomic DNA), RNA molecules (e.g., mRNA), natural occurring, mutated, synthetic DNA or RNA molecules, and analogues of the DNA or RNA generated using nucleotide analogues. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

As used herein the term “TONSOKU” is used to refer to the protein encoded by the “TONSOKU” gene. The term “TONSOKU” as used herein therefore encompasses the proteins encoded by the “TONSOKU”, “MGOUN3” or “BRUSHY1” genes referred to in the literature.

The TONSOKU protein sequence is well known by a person of skill in the art. By way of example only, the TONSOKU protein of A. thaliana has a sequence of SEQ ID NO: 1. SEQ ID NO:1 is therefore an example of an “endogenous TONSOKU protein” or “wildtype TONSOKU protein”. Other TONSOKU protein sequences found in plants are readily identifiable to a person of skill in the art. For the avoidance of doubt, the term TONSOKU therefore encompasses the sequence of SEQ ID NO:1 and plant homologues thereof.

Homologues of the plant protein are also known in animals, such as “TONSL” which is also known as “NFKBIL2” (O'Donnell et al., 2010). Such homologues are readily identifiable to a person of skill in the art. The invention is therefore not limited to TONSOKU, but may also apply to non-plant homologues thereof, such as those found in animals.

The terms “polypeptide” and “protein” are used interchangeably herein and refer to amino acids in a polymeric form of any length, linked together by peptide bonds.

Studies on mutant tonsoku⁻ plants have revealed that it is required for proper cell arrangement in root and shoot apical meristems (Suzuki et al., 2004; Guyomarc'h et al., 2004). It has also been found to be involved in chromatin dynamics and genome maintenance in plants (Guyomarc'h et al., 2006). It has been implicated in linking responses to DNA damage and gene silencing in plants (Takeda et al., 2004). Finally, the gene is known to be required for genome maintenance (Ohno et al., 2011).

The TONSOKU protein has been characterised as a nuclear protein with two predicted protein-protein (tetratricopeptide repeats (TPR) and (leucine rich repeats (LRR)) interaction domains (Takeda et al., 2004). The yeast homologue of TONSOKU protein is TONSL. The TONSL protein complexes with MMS22L and the complex mediates recovery from replication stress and homologous recombination (O'Donnell et al., 2010). Finally, it has recently been determined that H4Kme0 marks post-replicative chromatin and recruits the TONSL-MMS22L DNA repair complex (Saredi et al., 2016).

Bi-allelic variants in TONSL have also been implicated as the cause of diseases such as SPONASTRIME Dysplasia and a spectrum of skeletal dysplasia phenotypes in humans (Burrage et al., 2019).

The methods of the invention are described below in the context of the TONSOKU gene (which encompasses the gene of SEQ ID NO:3 and plant homologues thereof) and/or the TONSOKU protein (which encompasses the protein of SEQ ID NO:1 and plant homologues thereof). However, as would be clear to a person of skill in the art, the invention may also apply to non-plant homologues of the TONSOKU gene and/or the TONSOKU protein, such as those found in animals. Accordingly, all text below that relates to the TONSOKU gene and/or the TONSOKU protein applies equally to non-plant homologues thereof. In this context, throughout the text the terms “TONSOKU gene” and/or the “TONSOKU protein” may be replaced with “TONSOKU gene homologue” and/or the “TONSOKU protein homologue” respectively.

The methods of the invention all involve a step in which there is the reduction or abolition of the expression of at least one TONSOKU nucleic acid sequence and/or reduction or abolition of an activity of a TONSOKU polypeptide in a cell.

The term “reducing” means that there is a decrease in the levels of TONSOKU protein expression and/or TONSOKU protein level (e.g. concentration) and/or TONSOKU protein activity by up to 10%, 20%, 30%, 40%, 50%, 60%, 70%, 80% or 90%. The reduction in TONSOKU protein expression or TONSOKU protein level or TONSOKU protein activity can be measured relative to a control cell. The decrease can be by at least 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80% or 90% in comparison to a control cell.

The term “abolish” means that no expression of TONSOKU is detectable or that no functional TONSOKU polypeptide is produced or present in the cell. The abolition of TONSOKU nucleic acid or TONSOKU protein can be measured relative to a control cell as described herein.

A “control cell” as used herein is a cell which has not been modified according to the methods of the invention. Suitably, the control cell may not have reduced expression of a TONSOKU nucleic acid, reduced levels of a TONSOKU polypeptide and/or reduced activity of a TONSOKU polypeptide. In one example, the control cell may have been genetically modified (for example, in a region that is distinct from the TONSOKU locus). Suitably, the control cell could be a wild-type cell. The control cell is typically of the same species, preferably having the same genetic background as the modified cell. Suitably, the control cell has endogenous TONSOKU or wildtype TONSOKU. Suitably, the control cell has endogenous TONSOKU or wildtype TONSOKU. Suitably, the control cell has an endogenous TONSOKU protein, gene and optionally promoter sequence as described elsewhere herein.

Methods for determining the presence of the TONSOKU gene or level of TONSOKU gene expression in a cell would be well known to the skilled person. Examples include using PCR or RT-PCR to detect TONSOKU nucleic acids (e.g. DNA or RNA). Methods for determining the level of TONSOKU protein in a cell would also be well known to the skilled person. Examples include using western blotting techniques or protein mass spectrometry such as peptide mass fingerprinting.

The reduction or abolition of the expression of at least one TONSOKU nucleic acid sequence and/or reduction or abolition of an activity of a TONSOKU polypeptide may be due to mutation of a TONSOKU nucleic acid (e.g. the TONSOKU gene), wherein the mutation causes a reduction or abolition of the expression of the TONSOKU nucleic acid sequence and/or a reduction or abolition of an activity of the TONSOKU polypeptide.

Alternatively, the reduction or abolition of the expression of at least one TONSOKU nucleic acid sequence and/or reduction or abolition of an activity of a TONSOKU polypeptide may be achieved by means of an inhibitor, which directly acts on the TONSOKU gene (e.g. see below regarding gene silencing), or directly acts on the TONSOKU protein (e.g. see below regarding inhibitor molecules such as peptide inhibitors, antibodies etc). Inhibitors that directly act on the TONSOKU gene or TONSOKU protein may also be referred to as inhibitors that are specific for the TONSOKU gene or TONSOKU protein. Inhibitors that directly act on the TONSOKU gene or TONSOKU protein may bind directly to the TONSOKU gene or TONSOKU protein. Further details are provided below.

Accordingly, in one aspect, the step of reducing or abolishing the expression of at least one TONSOKU nucleic acid in a cell, can comprise introducing at least one mutation into the genome of said cell.

By “at least one mutation” it means that where the TONSOKU gene is present as more than one copy or homologue (with the same or slightly different sequence) there is at least one mutation in at least one gene or in a single copy of the gene (e.g. it is a heterozygous mutation of the TONSOKU gene). Alternatively, in for example a cell with a diploid genome, both copies of the TONSOKU gene may be mutated. Alternatively, in for example a cell with a polyploid genome, all copies of the gene can be mutated in the cell.

The method may comprise introducing at least one mutation into the endogenous TONSOKU gene and/or the TONSOKU gene promoter within the cell. Said mutation can be in the coding region of the TONSOKU gene. Alternatively, the at least one mutation may be introduced into the TONSOKU gene such that the altered gene does not express a full-length (in other words is a truncated form) TONSOKU protein or does not express a fully functional TONSOKU protein. In this manner, the activity of the TONSOKU polypeptide can be considered to be reduced or abolished as determined by methods described elsewhere herein. In any case, the mutation may result in the expression of TONSOKU with no, significantly reduced or altered biological activity in vivo. Alternatively, the TONSOKU protein may not be expressed at all.

Alternatively, at least one mutation or structural alteration may be introduced into the TONSOKU promoter such that the TONSOKU gene is either not expressed (in other words is abolished) or expression is reduced.

Suitably, the sequence of the TONSOKU promoter may comprise or consist of a nucleic acid sequence as defined in SEQ ID NO: 2.

Suitably, the sequence of the TONSOKU gene may comprise or consist of a nucleic acid sequence as defined in SEQ ID NO: 3 or SEQ ID NO: 4, which encodes a polypeptide as defined in SEQ ID NO: 1.

The term “endogenous” nucleic acid as described herein may refer to the native or natural sequence in the genome of the cell. The endogenous sequence of the TONSOKU gene can, for example, be defined as SEQ ID NO: 3, which encodes an amino acid sequence as defined in SEQ ID NO: 1.

Suitably, the mutation that is introduced into the endogenous TONSOKU gene or TONSOKU promoter thereof to reduce, or inhibit the biological activity and/or expression levels of the TONSOKU gene can be selected from the following mutation types:

a “missense mutation”, which is a change in the nucleic acid sequence that results in the substitution of an amino acid for another amino acid;
a “nonsense mutation” or “STOP codon mutation”, which is a change in the nucleic acid sequence that results in the introduction of a premature STOP codon and, thus, the termination of translation (resulting in a truncated protein);
an “insertion mutation” of one or more amino acids, due to one or more codons having been added in the coding sequence of the nucleic acid;
a “deletion mutation” of one or more amino acids, due to one or more codons having been deleted in the coding sequence of the nucleic acid;
a “frameshift mutation”, resulting in the nucleic acid sequence being translated in a different frame downstream of the mutation. A frameshift mutation can have various causes, such as the insertion, deletion or duplication of one or more nucleotides; and/or
a “splice site” mutation, which is a mutation that results in the insertion, deletion or substitution of a nucleotide at the site of splicing.

The skilled person will understand that at least one mutation as defined above and which leads to the insertion, deletion or substitution of at least one nucleic acid or amino acid compared to the wild-type TONSOKU promoter or TONSOKU nucleic acid or protein sequence can affect the biological activity of the TONSOKU protein.

The at least one mutation as described herein may alternatively be introduced into a regulatory element of the at least one TONSOKU gene. As used herein the term “regulatory element” is used to refer to regions of non-coding DNA which regulate the transcription of the TONSOKU gene. The regulatory element can either be a cis-regulatory element or a trans-regulatory element. Examples of cis-regulatory elements are enhancers, silencers and operators.

The TONSOKU genes in other plants may be identified by performing a BLAST alignment search with the TONSOKU sequence from Arabidopsis thaliana.

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or sub-sequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Suitable homologues can be identified by sequence comparisons and identifications of conserved domains. There are predictors in the art that can be used to identify such sequences. The function of the homologue can be identified as described herein and a skilled person would thus be able to confirm the function, for example when overexpressed in a plant.

Thus, the nucleotide sequences of the invention and described herein can also be used to isolate corresponding sequences from other organisms. This is particularly the case for other plants such as crop plants (which are defined elsewhere herein). Standard molecular techniques may be used to identify the TONSOKU gene from a particular plant species. For example, oligonucleotide probes based on the TONSOKU, MGOUN3 or BRUSHY1 plant sequences can be used to identify the desired polynucleotide in a cDNA or genomic DNA library from a desired plant species. Probes may be used to hybridize with genomic DNA or cDNA sequences to isolate homologous genes in the plant species of interest.

Alternatively, the TONSOKU gene can be amplified from nucleic acid samples using routine amplification techniques. For instance, PCR may be used to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. Appropriate primers and probes for identifying the TONSOKU gene in a plant can be generated based on the TONSOKU, MGOUN3 or BRUSHY1 plants' sequences. For a general overview of PCR see PCR Protocols: A Guide to Methods and Applications (Innis, M, Gelfand, D., Sninsky, J. and White, T., eds.), Academic Press, San Diego (1990).

In this manner, methods such as PCR, hybridization, and the like can be used to identify sequences based on their sequence homology to the sequences described herein. Topology of the sequences and the characteristic domains structure can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (e.g. genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

As described above, the methods described herein can comprise introducing at least one mutation into the endogenous TONSOKU gene and/or the TONSOKU promoter. Such mutations can be introduced by using mutagenesis or targeted genome editing. The resulting product of the methods described herein can be referred to as mutants or modified cells. Accordingly, the term “mutant” and “modified cell” are used interchangeably herein. The invention may therefore relate to a method in which the mutant described herein has been generated by genetic engineering methods and thus does not encompass naturally occurring varieties.

For plant cells in particular, conventional mutagenesis methods can be used to introduce at least one mutation into a TONSOKU gene or TONSOKU promoter sequence. These methods include both physical and chemical mutagenesis. A skilled person will know further approaches can be used to generate such mutants, and methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

Insertional mutagenesis can be used, for example using T-DNA mutagenesis (which inserts the T-DNA from the Agrobacterium tumefaciens Ti-Plasmid into DNA causing either loss of gene function (e.g. by mutation) or gain of gene function (e.g. by epigenetic effects)), site-directed nucleases (SDNs) or transposons as a mutagen. Insertional mutagenesis is based on the insertion of foreign DNA into the gene of interest (see Krysan et al, The Plant Cell, Vol. 11, 2283-2290, December 1999). Accordingly, T-DNA can be used as an insertional mutagen to disrupt the TONSOKU gene or TONSOKU promoter expression in plant cells. T-DNA not only disrupts the expression of the gene into which it is inserted, but also acts as a marker for subsequent identification of the mutation. Since the sequence of the inserted element is known, the gene in which the insertion has occurred can be recovered, using various cloning or PCR-based strategies. The insertion of a piece of T-DNA in the order of 5 to 25 kb in length generally produces a disruption of gene function. If a large enough population of T-DNA transformed lines is generated, there are reasonably good chances of finding a transgenic plant carrying a T-DNA insert within the TONSOKU gene or TONSOKU promoter. Transformation of cells with T-DNA is achieved by an Agrobacterium-mediated method which involves exposing plant cells and tissues to a suspension of Agrobacterium cells.

The details of this method are well known to a skilled person. In short, plant transformation by Agrobacterium results in the integration into the nuclear genome of a sequence called T-DNA, which is carried on a bacterial plasmid. The use of T-DNA transformation leads to stable single insertions. Further mutant analysis of the resultant transformed lines is straightforward and each individual insertion line can be rapidly characterized by direct sequencing and analysis of DNA flanking the insertion. Gene expression in the mutant is compared to expression of the TONSOKU nucleic acid sequence in a wild type plant and phenotypic analysis is also carried out.

Alternatively, the mutagenesis employed can be a type of physical mutagenesis, such as application of ultraviolet radiation, X-rays, gamma rays, fast or thermal neutrons or protons. The targeted population can then be screened to identify a TONSOKU loss of function mutant.

As a further alternative, the method may comprise mutagenizing a plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7, 12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde.

Another alternative method that can be used to create and analyse mutations in whole plants is targeting induced local lesions in genomes (TILLING), reviewed in Henikoff et al, 2004. In this method, seeds are mutagenised with a chemical mutagen, for example EMS. The resulting M1 plants are self-fertilised and the M2 generation of individuals is used to prepare DNA samples for mutational screening. DNA samples are pooled and arrayed on microtiter plates and subjected to gene specific PCR. The PCR amplification products may be screened for mutations in the TONSOKU target gene using any method that identifies heteroduplexes between wild type and mutant genes. For example, but not limited to, denaturing high pressure liquid chromatography (dHPLC), constant denaturant capillary electrophoresis (CDCE), temperature gradient capillary electrophoresis (TGCE), or by fragmentation using chemical cleavage. Preferably the PCR amplification products are incubated with an endonuclease that preferentially cleaves mismatches in heteroduplexes between wild type and mutant sequences. Cleavage products are electrophoresed using an automated sequencing gel apparatus, and gel images are analyzed with the aid of a standard commercial image-processing program. Any primer specific to the TONSOKU nucleic acid sequence may be utilized to amplify the TONSOKU nucleic acid sequence within the pooled DNA sample. Preferably, the primer is designed to amplify the regions of the TONSOKU gene where useful mutations are most likely to arise. To facilitate detection of PCR products on a gel, the PCR primer may be labelled using any conventional labelling method.

Rapid high-throughput screening procedures thus allow the analysis of amplification products for identifying a mutation conferring the reduction or inactivation of the expression of the TONSOKU gene as compared to a corresponding non-mutagenised wild type plant. Once a mutation is identified in a gene of interest, the seeds of the M2 plant carrying that mutation are grown into adult M3 plants and screened for the phenotypic characteristics associated with the target gene TONSOKU. Loss of and reduced function mutants with increased endogenous tandem duplication(s) as compared to a control plant can thus be identified.

The above described methods are typically used to mutagenize plants. Other mutagenesis methods that are not plant specific are well known in the art. These methods can comprise introducing at least one mutation into the endogenous TONSOKU gene and/or the TONSOKU promoter into a cell. One example of this is the introduction of mutations by targeted genome editing.

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customisable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of FokI to direct nucleolytic activity toward specific genomic loci.

Upon delivery into host cells via the bacterial type III secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD that determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the FokI nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471. Cermak T et al. describes a set of customized plasmids that can be used with the Golden Gate cloning method to assemble multiple DNA fragments. As described therein, the Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct. Accordingly, using techniques known in the art it is possible to design a TAL effector that targets a TONSOKU gene or promoter sequence as described herein.

Another genome editing method that can be used is CRISPR. The use of this technology in genome editing is well described in the art, for example in U.S. Pat. No. 8,697,359 and references cited herein. In short, CRISPR is a microbial nuclease system involved in defence against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA).

CRISPR/Cas can also be used to modulate gene expression by using modified “dead” Cas proteins fused to transcriptional activational domains (see, e.g., Khatodia et al. Frontiers in Plant Science 2016 7: article 506 for a review of CRISPR technology). The Cas protein may be a type I, type II, type III, type IV, type V, or type VI Cas protein. The Cas protein may comprise one or more domains. Non-limiting examples of domains include, a guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. The guide nucleic acid recognition and/or binding domain may interact with a guide nucleic acid. In some embodiments, the nuclease domain may comprise one or more mutations resulting in a nickase or a “dead” enzyme (e.g. the nuclease domain lacks catalytic activity).

Cas proteins include c2c1, C2c2, c2c3, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Cas9 (Csn1 or Csx12), Cas10, Cas10d, Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof.

The most widely used Cas protein for techniques using CRISPR/Cas technology is Cas9. Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, for example, sgRNAs have been expressed using plant RNA polymerase III promoters, such as U6 and U3. Accordingly, using techniques known in the art it is possible to design sgRNA molecules that targets a TONSOKU gene or TONSOKU promoter sequence as described herein.

Cas9 expression plasmids for use in the methods of the invention can be constructed as described in the art.

Whilst the above described methods are directed to mutation of a nucleic acid sequence (such as a gene or promoter), the methods described herein also encompass the reduction of expression of the TONSOKU gene at either the level of transcription or translation.

For example, expression of a TONSOKU nucleic acid sequence, as defined elsewhere herein, can be reduced or silenced using a number of gene silencing methods known to the skilled person, such as, but not limited to, the use of small interfering nucleic acids (siNA) against TONSOKU. “Gene silencing” is a term generally used to refer to suppression of expression of a gene via sequence-specific interactions that are mediated by RNA molecules. The degree of reduction may be so as to totally abolish production of the encoded gene product, but more usually the abolition of expression is partial, with some degree of expression remaining. The term should not therefore be taken to require complete “silencing” of expression.

The siNAs may include, short interfering RNA (siRNA), double-stranded RNA (dsRNA), micro-RNA (miRNA), antagomirs and short hairpin RNA (shRNA) capable of mediating RNA interference.

The reduction of expression of the TONSOKU gene at either the level of transcription or translation inhibition can be measured by determining the presence and/or amount of TONSOKU transcript using techniques well known to the skilled person (such as Northern Blotting, RT-PCR).

Moreover, transgenes may be used to suppress endogenous genes. Many, if not all, genes can be “silenced” by transgenes. Gene silencing requires sequence similarity between the transgene and the gene that becomes silenced. This sequence homology may involve promoter regions or coding regions of the silenced target gene. When coding regions are involved, the transgene able to cause gene silencing may have been constructed with a promoter that would transcribe either the sense or the antisense orientation of the coding sequence RNA. It is likely that the various examples of gene silencing involve different mechanisms that are not well understood. In different examples there may be transcriptional or post-transcriptional gene silencing and both may be used according to the methods of the invention. The mechanisms of gene silencing and their application in genetic engineering, which were first discovered in plants in the early 1990s and then shown in C. elegans are extensively described in the literature. RNA-mediated gene suppression or RNA silencing according to the methods of the invention includes co-suppression wherein over-expression of the target sense RNA or mRNA, that is the TONSOKU sense RNA or mRNA, leads to a reduction in the level of expression of the genes concerned. RNAs of the transgene and homologous endogenous gene are co-ordinately suppressed. Other techniques used in the methods described herein include antisense RNA to reduce transcript levels of the endogenous target gene in a cell. In this method, RNA silencing does not affect the transcription of a gene locus, but only causes sequence-specific degradation of target mRNAs. An “antisense” nucleic acid sequence comprises a nucleotide sequence that is complementary to a “sense” nucleic acid sequence encoding a TONSOKU protein, or a part of the protein, e.g. complementary to the coding strand of a double-stranded cDNA molecule or complementary to an mRNA transcript sequence. The antisense nucleic acid sequence is preferably complementary to the endogenous TONSOKU gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions). Antisense nucleic acid sequences can be designed according to the rules of Watson and Crick base pairing. The antisense nucleic acid sequence may be complementary to the entire TONSOKU nucleic acid sequence as defined herein, but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine-substituted nucleotides may be used. Examples of modified nucleotides that may be used to generate the antisense nucleic acid sequences are well known in the art. The antisense nucleic acid sequence can be produced biologically using an expression vector into which a nucleic acid sequence has been subcloned in an antisense orientation (e.g. RNA transcribed from the inserted nucleic acid will be of an antisense orientation to a target nucleic acid of interest). Preferably, production of antisense nucleic acid sequences in cells occurs by means of a stably integrated nucleic acid construct comprising a promoter, an operably linked antisense oligonucleotide, and a terminator. The nucleic acid molecules used for silencing in the methods of the invention hybridize with or bind to mRNA transcripts and/or insert into genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a cell by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using vectors.

RNA interference (RNAi) is another post-transcriptional gene-silencing phenomenon which may be used according to the methods of the invention. This is induced by double-stranded RNA in which mRNA that is homologous to the dsRNA is specifically degraded. It refers to the process of sequence-specific post-transcriptional gene silencing mediated by short interfering RNAs (siRNA). The process of RNAi begins when the enzyme, DICER, encounters dsRNA and chops it into pieces called small-interfering RNAs (siRNA). This enzyme belongs to the RNase III nuclease family. A complex of proteins gathers up these RNA remains and uses their code as a guide to search out and destroy any RNAs in the cell with a matching sequence, such as target mRNA.

Artificial and/or natural microRNAs (miRNAs) may be used to knock out gene expression and/or mRNA translation. MicroRNAs (miRNAs) miRNAs are typically single stranded small RNAs typically 19-24 nucleotides long. Most miRNAs have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. miRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes. Artificial microRNA (amiRNA) technology has been applied in Arabidopsis thaliana and other plants to efficiently silence target genes of interest. The design principles for amiRNAs have been generalized and integrated into a Web-based tool (http://wmd.weigelworld.org).

Thus, a cell may be transformed to introduce a RNAi, shRNA, snRNA, dsRNA, siRNA, miRNA, ta-siRNA, amiRNA or co-suppression molecule that has been designed to target the expression of a TONSOKU nucleic acid sequence and selectively decreases or inhibits the expression of the gene or stability of its transcript. The RNAi, snRNA, dsRNA, shRNA siRNA, miRNA, amiRNA, ta-siRNA or co-suppression molecule used may comprise a fragment of at least 17 nt, preferably 22 to 26 nt and can be designed on the basis of the information shown in any of SEQ ID NOs. 3 or 4. Guidelines for designing effective siRNAs are known to the skilled person. Briefly, a short fragment of the target gene sequence (e.g., 19-40 nucleotides in length) is chosen as the target sequence of the siRNA of the invention. The short fragment of target gene sequence is a fragment of the target gene mRNA. The criteria for choosing a sequence fragment from the target gene mRNA to be a candidate siRNA molecule include 1) a sequence from the target gene mRNA that is at least 50-100 nucleotides from the 5′ or 3′ end of the native mRNA molecule, 2) a sequence from the target gene mRNA that has a G/C content of between 30% and 70%, most preferably around 50%, 3) a sequence from the target gene mRNA that does not contain repetitive sequences (e.g., AAA, CCC, GGG, TTT etc), 4) a sequence from the target gene mRNA that is accessible in the mRNA, 5) a sequence from the target gene mRNA that is unique to the target gene, 6) avoids regions within 75 bases of a start codon. The sequence fragment from the target gene mRNA may meet one or more of the criteria identified above. The selected gene is introduced as a nucleotide sequence in a prediction program that takes into account all the variables described above for the design of optimal oligonucleotides. This program scans any mRNA nucleotide sequence for regions susceptible to be targeted by siRNAs. The output of this analysis is a score of possible siRNA oligonucleotides. The highest scores are used to design double stranded RNA oligonucleotides that are typically made by chemical synthesis. In addition to siRNA which is complementary to the mRNA target region, degenerate siRNA sequences may be used to target homologous regions. siRNAs according to the invention can be synthesized by any method known in the art. RNAs are preferably chemically synthesized using appropriately protected ribonucleoside phosphoramidites and a conventional DNA/RNA synthesizer. Additionally, siRNAs can be obtained from commercial RNA oligonucleotide synthesis suppliers. siRNA molecules according to the aspects of the invention may be double stranded. Double stranded siRNA molecules may comprise blunt ends. Alternatively, double stranded siRNA molecules may comprise overhanging nucleotides (e.g., 1-5 nucleotide overhangs, preferably 2 nucleotide overhangs). The siRNA could be a short hairpin RNA (shRNA); and the two strands of the siRNA molecule may be connected by a linker region (e.g., a nucleotide linker or a non-nucleotide linker). The siRNAs may contain one or more modified nucleotides and/or non-phosphodiester linkages. Chemical modifications well known in the art are capable of increasing stability, availability, and/or cell uptake of the siRNA. The skilled person will be aware of other types of chemical modification which may be incorporated into RNA molecules. Recombinant DNA constructs as described in U.S. Pat. No. 6,635,805, may be used.

Conventional methods, such as a vector and Agrobacterium-mediated transformation, are used for introduction of the silencing RNA molecule into a plant cell. Stably transformed plant cells can thus be generated and expression of the TONSOKU gene compared to a wild type control plant can be analysed.

Silencing of the TONSOKU nucleic acid sequence may also be achieved using virus-induced gene silencing.

Thus, the plant may express a nucleic acid construct comprising a RNAi, shRNA snRNA, dsRNA, siRNA, miRNA, ta-siRNA, amiRNA or co-suppression molecule that targets the TONSOKU nucleic acid sequence as described herein and reduces expression of the endogenous TONSOKU nucleic acid sequence. A gene is targeted when, for example, the RNAi, snRNA, dsRNA, siRNA, shRNA miRNA, ta-siRNA, amiRNA or co-suppression molecule selectively decreases or inhibits the expression of the gene compared to a control cell. Alternatively, a RNAi, snRNA, dsRNA, siRNA, miRNA, ta-siRNA, amiRNA or co-suppression molecule targets a TONSOKU nucleic acid sequence when the RNAi, shRNA snRNA, dsRNA, siRNA, miRNA, ta-siRNA, amiRNA or co-suppression molecule hybridises under stringent conditions to the gene transcript.

A further approach to gene silencing is by targeting nucleic acid sequences complementary to the regulatory region of the gene (e.g., the promoter and/or enhancers) of TONSOKU to form triple helical structures that prevent transcription of the gene in target cells.

The suppressor nucleic acids may be anti-sense suppressors of expression of the TONSOKU polypeptides. In using anti-sense sequences to down-regulate gene expression, a nucleotide sequence is placed under the control of a promoter in a “reverse orientation” such that transcription yields RNA which is complementary to normal mRNA transcribed from the “sense” strand of the target gene. An anti-sense suppressor nucleic acid may comprise an anti-sense sequence of at least 10 nucleotides from the target nucleotide sequence. It may be preferable that there is complete sequence identity in the sequence used for down-regulation of expression of a target sequence, and the target sequence, although total complementarity or similarity of sequence is not essential. One or more nucleotides may differ in the sequence used from the target gene. Thus, a sequence employed in a down-regulation of gene expression in accordance with the present invention may be a wild-type sequence (e.g. gene) selected from those available, or a variant of such a sequence. The sequence need not include an open reading frame or specify an RNA that would be translatable. It may be preferred for there to be sufficient homology for the respective anti-sense and sense RNA molecules to hybridise. There may be down regulation of gene expression even where there is about 5%, 10%, 15% or 20% or more mismatch between the sequence used and the target gene. Effectively, the homology should be sufficient for the down-regulation of gene expression to take place.

Nucleic acid which suppresses expression of a TONSOKU polypeptide as described herein may be operably linked to a heterologous regulatory-sequence, such as a promoter, for example a constitutive, inducible, tissue-specific or developmental specific promoter. The construct or vector may be transformed into cells and expressed as described herein.

Cells comprising such vectors are also within the scope of the invention. Also encompassed are silencing construct obtainable or obtained by a method as described herein and to cell comprising such construct.

In summary, methods for decreasing or abolishing TONSOKU expression involve targeted mutagenesis methods, specifically genome editing, and exclude methods that are solely based on generating plants by traditional breeding methods.

The methods described herein up until this point are directed to reducing or abolishing TONSOKU nucleic acid expression. In another aspect of the invention, the method can reduce or abolish an activity of a TONSOKU polypeptide in a cell.

In particular, it can be envisaged that synthetic (e.g. man-made) molecules may be useful for inhibiting the biological function of a TONSOKU polypeptide, or for interfering with the signalling pathway in which the TONSOKU polypeptide is involved. These synthetic molecules can be characterised by their ability to bind to a TONSOKU polypeptide. Therefore, TONSOKU activity can be reduced by providing the cell with a TONSOKU binding molecule. The activity of TONSOKU can be reduced by at least 30%, 40%, 50%, 60%, 70%, 80%, 90%, 95% or 100% as compared to a corresponding wild-type cell. The TONSOKU binding molecule can bind to TONSOKU and inhibit its enzyme activity. Alternatively, the TONSOKU binding molecule may inhibit its ability to bind to other proteins. In one example, the TONSOKU binding molecule may in itself be a peptide inhibitor.

Additional binding agents include antibodies as well as non-immunoglobulin binding agents, such as phage display-derived peptide binders, and antibody mimics, e.g., affibodies, tetranectins (CTLDs), adnectins (monobodies), anticalins, DARPins (ankyrins), avimers, iMabs, microbodies, peptide aptamers, Kunitz domains, aptamers and affilins. For example, antibodies (or other binding agents) directed to an endogenous TONSOKU polypeptide can be used for inhibiting its function in vitro or in vivo. Alternatively, the antibody can be used for interfering with the signalling pathway in which a TONSOKU polypeptide is involved.

The term “antibody” includes, for example, both naturally occurring and non-naturally occurring antibodies, polyclonal and monoclonal antibodies, chimeric antibodies and wholly synthetic antibodies and fragments thereof, such as, for example, the Fab′, F(ab′)2, Fv or Fab fragments, or other antigen recognizing immunoglobulin fragments.

Antibodies which bind a particular epitope can be generated by methods known in the art. For example, polyclonal antibodies can be made by the conventional method of immunizing a mammal (e.g., rabbits, mice, rats, sheep, goats). Polyclonal antibodies are then contained in the sera of the immunized animals and can be isolated using standard procedures (e.g., affinity chromatography, immunoprecipitation, size exclusion chromatography, and ion exchange chromatography). Monoclonal antibodies can be made by the conventional method of immunization of a mammal, followed by isolation of plasma B cells producing the monoclonal antibodies of interest and fusion with a myeloma cell (see, e.g., Mishell, et al., 1980). Screening for recognition of the epitope can be performed using standard immunoassay methods including ELISA techniques, radioimmunoassays, immunofluorescence, immunohistochemistry, and Western blotting. In vitro methods of antibody selection, such as antibody phage display, may also be used to generate antibodies (see, e.g., Schirrmann et al. 2011). A nuclear localization signal can also be added to the antibody in order to increase localization to the nucleus.

Cells comprising an inhibitor of the biological function of a TONSOKU polypeptide, or an inhibitor for interfering with the signalling pathway in which the TONSOKU polypeptide is involved are also encompassed within the invention.

The methods described herein are directed to reducing or abolishing TONSOKU nucleic acid expression or reducing or abolishing the presence of TONSOKU polypeptide or reducing or abolishing an activity of a TONSOKU polypeptide in a cell.

A cell as described herein refers to any cell type. As stated elsewhere herein the invention has utility in plant and animal cells. Accordingly, the cell can be a mammalian cell, for example. Alternatively, the cell can be a plant cell. The term “plant cell” also encompasses, suspension cultures, callus tissue, embryos, meristematic regions, gametophytes, sporophytes, pollen and microspores. The plant cell as described herein can be a plant cell from a crop plant.

The reduction or abolition of a TONSOKU nucleic acid or TONSOKU protein has been found by the inventors to increase the endogenous genome modification in a cell. Thus, the invention provides a novel method of increasing endogenous genome modification in a cell.

The term “increase” is defined herein as an elevation of endogenous genome modification. The increase can be measured relative to a control cell as defined elsewhere herein. The increase in endogenous genome modification can be by at least 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90% or 100% in comparison to a control cell.

The term “genome modification” is defined herein to refer to any type of alteration within the genomic content of a plant cell. For example, genome modification includes insertion, modification, deletion or replacement of portions of the genome of a cell. It has been found that the methods of the invention are particularly useful for increasing endogenous insertions within the genome of a cell.

The term “endogenous genome modification” is defined herein as naturally occurring genome modification events taking place within a cell such as via natural recombination. This contrasts with genetic engineering methods, for example, which involve application of exogenous compositions to a plant cell in order to artificially modify the genome of the plant cell. In other words, endogenous genome modification encompasses non-transgenic genome modification.

The inventors have observed an increase in tandem duplications in cells that have been subjected to the methods described herein. Tandem duplication events result in insertions within the genome of a cell wherein the insertion is one or more repeated unit(s) of a sequence that is already in the genome of the cell. The tandem duplication event results in repeated units that are in tandem within the genome which may therefore be referred to as a “tandem duplication”. In other words, tandem duplication events result in a genome with a pattern of nucleotides (in this case a “unit sequence”) repeated, wherein the repetitions are directly adjacent to each other, generating a tandem duplication. A tandem duplication event may introduce at least one unit sequence, for example, it may introduce at least two, at least three etc unit sequences into the genome. A tandem duplication is therefore not limited to two unit sequences directly adjacent to each other; it encompasses any number of repeated unit sequences in tandem. For the avoidance of doubt, “tandem duplication event(s)” is used herein to refer to a process step and “tandem duplication(s)” is used herein to refer to the product of the process step e.g. the resulting modification within the genome resulting from the tandem duplication event.

The number of repetitions of the unit sequence within the tandem duplication is referred to herein as the number of “tandem repeats”. By way of an example, if the unit sequence is ATTCG (SEQ ID NO: 5), a polynucleotide comprising two tandem repeats of the unit sequence would comprise the sequence ATTCGATTCG (SEQ ID NO: 6), a polynucleotide comprising three tandem repeats of the unit sequence would comprise the sequence ATTCGATTCGATTCG (SEQ ID NO: 7), a polynucleotide comprising four tandem repeats of the unit sequence would comprise the sequence ATTCGATTCGATTCGATTCG (SEQ ID NO: 8) etc. The number of tandem repeats can also be referred to as the “copy number” of the unit sequence.

The methods described herein can introduce a plurality of tandem duplications into the genome at different genomic locations. In other words, more than one unit sequence can be duplicated within the genome. In this context, each set of repetitions of a unit sequence within the genome is referred to herein as a “tandem duplication”. The terms “tandem duplication” and “tandem duplications” are used interchangeably herein and use of each of said terms encompasses both a single tandem duplication and a plurality of tandem duplications. By way of an example, if one unit sequence is duplicated (e.g. ATTCG (SEQ ID NO: 5)), a second unit sequence that is independent of the first unit sequence may also be duplicated (e.g. TATACAG (SEQ ID NO: 9)) within the same genome. The number of tandem repeats of each unit sequence can be different. By way of an example, the genome may comprise three tandem repeats of ATTCG (SEQ ID NO: 5) and additionally may comprise two tandem repeats of TATACAG (SEQ ID NO: 9) within said genome. In the above example, the number of tandem duplications in the genome is two.

FIG. 2 shows conceptual examples of genomes that are WT as well as modified by the methods described herein. In one instance, the methods described herein results in a single tandem duplication, where a duplication event results in two copies of the unit sequence (e.g. two tandem repeats) within one tandem duplication (FIG. 2A). In another instance, the methods described herein results in a plurality of tandem duplications (e.g. two tandem duplications), wherein one of the duplication events results in two copies of the unit sequence (i.e. two tandem repeats) within one tandem duplication and another tandem duplication event results in three copies of the unit sequence (e.g. three tandem repeats) in a distinct tandem duplication (FIG. 2B). The methods described herein may introduce said tandem duplications via sequential processes (e.g. the induction of a first tandem duplication event followed by induction of a second tandem duplication event). Alternatively, the methods described herein may introduce a plurality of tandem duplications via a single step (e.g. the induction of a first tandem duplication event and a second tandem duplication event simultaneously).

The number of tandem duplications in the genome introduced by the methods described herein can, for example be about 1, 2, 3, 4, 5, 6, 7, 8, 9 or 10. Alternatively, the number of tandem duplications can be at least about 10, 11, 12, 13, 14, 15, 16, 17, 18, 19 or 20. Alternatively, the number of tandem duplications can be at least about 10, 15, 20, 25, 30, 45, 50, 55, 60, 65, 70, 75, 80, 85, 90, 95 or 100.

The number of tandem repeats within the at least one tandem duplication within the genome by the methods described herein can be at least about 2, 3, 4, 5, 6, 7, 8, 9 or 10. Alternatively, the number of tandem repeats can be at least about 10, 11, 12, 13, 14, 15, 16, 17, 18, 19 or 20. Alternatively, the number of tandem repeats can be at least about 10, 15, 20, 25, 30, 45, 50, 55, 60, 65, 70, 75, 80, 85, 90, 95 or 100.

In the methods described herein the unit sequence is from about 30 to about 3000 kilobases. The unit sequence may therefore be from about 30 to about 2500 kilobases. The unit sequence may therefore be from about 30 to about 2000 kilobases. The unit sequence may therefore be from about 30 to about 1500 kilobases. The unit sequence may therefore be from about 30 to about 1000 kilobases. The unit sequence may therefore be from about 30 to about 500 kilobases.

The unit sequence may be from about 50 to about 500 kilobases long. The unit sequence may therefore comprise at least about 50, 100, 150, 200, 250, 300, 350, 400, 450 or 500 kilobases (with the upper limit for each case being about 500 kilobases). Therefore, a unit sequence may for example be, from about 50 to 100, from about 50 to 150, from about 50 to 200, from about 50 to 250, from about 50 to 300, from about 50 to 350, from about 50 to 400 or from about 50 to 450 kilobases. Alternatively, a unit sequence may for example be, from about 100 to 150, from about 100 to 200, from about 100 to 250, from about 100 to 300, from about 100 to 350, from about 100 to 400 or from about 100 to 450 kilobases. Alternatively, a unit sequence may for example be, from about 150 to 200, from about 150 to 250, from about 150 to 300, from about 150 to 350, from about 150 to 400 or from about 150 to 450 kilobases. Alternatively, a unit sequence may for example be, from about 200 to 250, from about 200 to 300, from about 200 to 350, from about 200 to 400 or from about 200 to 450 kilobases. Alternatively, a unit sequence may for example be, from about 250 to 300, from about 250 to 350, from about 250 to 400 or from about 250 to 450 kilobases. Alternatively, a unit sequence may for example be, from about 300 to 350, from about 300 to 400 or from about 300 to 450 kilobases. Alternatively, a unit sequence may for example be, from about from about 350 to 400 or from about 350 to 450 kilobases. Alternatively, a unit sequence may for example be, from about 400 to 450 kilobases. Alternatively, a unit sequence may for example be, from about from about 450 to 500 kilobases. A unit sequence of 50 to 500 kilobases can comprise a plurality of genes. Therefore, the invention provides a method of increasing the copy number of a plurality of genes within the genome. In this context, the plurality of genes are positioned proximally relative to one another within a chromosome of a cell.

Therefore, the methods described herein may introduce at least about 2, 3, 4, 5, 6, 7, 8, 9 or 10 tandem repeats wherein the unit sequence is from about 50 to about 500 kilobases long. Specifically, the methods described herein may introduce about 2 tandem repeats wherein the unit sequence is from about 50 to about 500 kilobases long. Specifically, the methods described herein may introduce about 3 tandem repeats wherein the unit sequence is from about 50 to about 500 kilobases long. Specifically, the methods described herein may introduce about 4 tandem repeats wherein the unit sequence is from about 50 to about 500 kilobases long. Specifically, the methods described herein may introduce about 5 tandem repeats wherein the unit sequence is from about 50 to about 500 kilobases long. Specifically, the methods described herein may introduce about 6 tandem repeats wherein the unit sequence is from about 50 to about 500 kilobases long. Specifically, the methods described herein may introduce about 7 tandem repeats wherein the unit sequence is from about 50 to about 500 kilobases long. Specifically, the methods described herein may introduce about 8 tandem repeats wherein the unit sequence is from about 50 to about 500 kilobases long. Specifically, the methods described herein may introduce about 9 tandem repeats wherein the unit sequence is from about 50 to about 500 kilobases long. Specifically, the methods described herein may introduce about 10 tandem repeats wherein the unit sequence is from about 50 to about 500 kilobases long.

The methods described herein increase the number of tandem repeats of the unit sequence within the genome of a cell. Whilst tandem duplication events are known to occur naturally in genomic DNA they typically occur at a very low level. Recent studies in C. elegans have observed that the CNV (copy number variants) rate in the order of 10⁻³ duplications/generation. In other words, in a population of 10 00 C. elegans worms, one C. elegans worm will have a gene duplication. In contrast, by using the methods described herein, the inventors have observed that the CNV rate in C. elegans will increase to approximately 0.75 duplication/generation in tnsl-1 deficient C. elegans.

The location at which the tandem duplication event(s) is induced by the methods described herein are at random e.g. indiscriminate. In other words, the increase in tandem duplication events occur within the genome at any location, irrespective of chromatin structure.

The tandem duplications produced by the methods described herein typically comprise at least two tandem repeats at a given genomic location within a cell. However, multiple tandem duplications have been observed at different genomic locations within the cell when the cell is grown for multiple generations. For example, at a first duplication stage one tandem duplication may be introduced into the genome of a cell, followed by a subsequent (or second) duplication stage in which a further tandem duplication is introduced into a different location as compared to the first tandem duplication, and so on.

The methods described herein comprise reducing or abolishing the expression of at least one TONSOKU nucleic acid sequence and/or reducing or abolishing the level of a TONSOKU polypeptide and/or reducing or abolishing an activity of a TONSOKU polypeptide in a cell, where the cells may be regenerated to whole organisms using standard techniques known in the art.

Plant cells are preferred in the methods described herein. Modified plant cells generated by the methods described herein are preferably identified by selection or screening and cultured in an appropriate medium that supports regeneration, can then be allowed to regenerate into plants. “Regeneration” refers to the process of growing a plant from a plant cell (e.g., plant protoplast or explant) and such methods are well-known in the art.

The plant cell or regenerated plant may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed plants may take a variety of forms. For example, they may be chimeras of transformed cells and non-transformed cells; clonal transformants (e.g., all cells transformed contain a desired mutation); grafts of transformed and untransformed tissues (e.g., in plants, a transformed rootstock grafted to an untransformed scion). Rapid high-throughput screening procedures allow the analysis of amplification products for identifying a mutation conferring the reduction or inactivation of the expression of the TONSOKU gene as compared to a corresponding non-mutagenised wild type plant.

Once a mutation is identified in a gene of interest, the seeds of the M2 plant carrying that mutation are grown into adult M3 plants and screened for the phenotypic characteristics associated with the target gene (e.g. TONSOKU). Loss of and reduced function mutants with increased endogenous tandem duplications compared to a control can thus be identified.

The methods as described herein can be employed in whole organisms, excluding humans. In preferred aspects, the methods as described herein are conducted in plants. Therefore, in addition to increasing tandem duplication events in in vitro cultivated plant cells, tissues or organs; an increase in tandem duplication events in whole living plants can also be achieved by the methods as described herein. Agrobacterium-mediated transfer is a widely applicable system for introducing nucleic acids into plant cells because the DNA can be introduced into whole plant tissues. Suitable processes include dipping of seedlings, leaves, roots, cotyledons, etc. in an Agrobacterium suspension which may be enhanced by vacuum-infiltration as well as for some plants the dipping of a flowering plant into an Agrobacteria solution (floral dip), followed by breeding of the transformed gametes.

The invention further provides a plant obtained or obtainable by the above described methods. For the purposes of the invention, a “genetically altered plant” or “mutant plant” is a plant that has been genetically altered compared to the naturally occurring wild type plant. A mutant plant is a plant that has been altered compared to the naturally occurring wild type plant using a mutagenesis method, such as any of the mutagenesis methods described herein. The mutagenesis method can for example be a targeted genome modification or genome editing. The plant genome can be altered compared to wild type sequences using a mutagenesis method. Such plants have an altered phenotype as described herein, such as an increased endogenous tandem duplications. Therefore, in this example, increased endogenous tandem duplications are conferred by the presence of an altered plant genome, for example, a mutated endogenous TONSOKU gene or TONSOKU promoter sequence. The endogenous promoter or gene sequence is specifically targeted using targeted genome modification and the presence of a mutated gene or promoter sequence is not conferred by the presence of transgenes expressed in the plant. In other words, the genetically altered plant can be described as transgene-free.

A plant according to the invention, including the transgenic plants, methods and uses described herein may be a monocot or a dicot plant. Preferably, the plant is a crop plant. By “crop plant” it is meant any plant which is grown on a commercial scale for human or animal consumption or use. Non-limiting examples include cotton, cantaloupe, radicchio, papaya, plum, peanut, oilseed rape, canola, sunflower, safflower, olive, sesame, hazelnut, almond, avocado, bay, pumpkin/squash, linseed, soya, pistachio, borage, maize, wheat, rye, oats, sorghum and millet, triticale, rice, barley, cassava, potato, sugarbeet, egg plant, alfalfa, perennial grasses, forage plants, oil palm, vegetables (brassicas, root vegetables, tuber vegetables, pod vegetables, fruiting vegetables, onion vegetables, leafy vegetables and stem vegetable), buckwheat, Jerusalem artichoke, broad bean, vetches, lentil, dwarf bean, lupin, clover, lucerne, tobacco, tomato, ornamental plants and cannabis (including marijuana and hemp).

As used herein, ornamental plants are plants that are grown for decorative and display purposes. For example, ornamental plants are grown in gardens and landscape design projects, as houseplants, cut flowers and specimen display.

Alternatively, the plant is Arabidopsis.

The term “plant” as used herein encompasses whole plants, ancestors and progeny of the plants and plant parts, including seeds, fruit, shoots, stems, leaves, roots (including tubers), flowers, tissues and organs. The term “plant” also encompasses plant cells, suspension cultures, callus tissue, embryos, meristematic regions, gametophytes, sporophytes, pollen and microspores.

One particular advantage associated with the methods described herein is that they can be used to generate a plant comprising at least one tandem duplication within the genome of the plant. The at least one tandem duplication can lead to the resulting plant exhibiting a new trait of interest that was not present in the wild type plant. The resulting plant can subsequently be screened for a trait of interest. In this manner, the methods described herein can be used for plant genetic engineering.

As used herein, a “trait” refers to the phenotype conferred from a particular gene or grouping of genes. A trait gene of interest includes any one gene or grouping of genes that encodes a trait. The terms “desired trait” and “trait of interest” are used interchangeably herein. Examples of traits that can be desired for plant genetic engineering purposes include insect resistance, disease resistance, herbicide tolerance, male sterility, abiotic stress tolerance, altered phosphorus utilisation, altered antioxidants, altered fatty acids, altered essential amino acids, altered carbohydrates, altered sequences involved in site-specific recombination, altered development, or altered morphology (such as size and pigmentation).

Further examples of traits of interest include an increase in yield, grain quality, nutrient content, starch quality and quantity, nitrogen fixation and/or utilization, and oil content and/or composition. The traits of interest can therefore improve crop yield, improve the desirability of crops, confer resistance to abiotic stress, such as drought, nitrogen, temperature, salinity, toxic metals or trace elements, or confer resistance to toxins such as pesticides and herbicides, or to biotic stress, such as attacks by fungi, viruses, bacteria, insects, and nematodes, and development of diseases associated with these organisms.

A trait that can be desired is insect resistance. A trait that can be desired is disease resistance. A trait that can be desired is herbicide tolerance. A trait that can be desired is male sterility. A trait that can be desired is abiotic stress tolerance. A trait that can be desired is altered phosphorus utilisation. A trait that can be desired is altered antioxidants. A trait that can be desired is altered fatty acids. A trait that can be desired is altered essential amino acids. A trait that can be desired is altered carbohydrates. A trait that can be desired is altered sequences involved in site-specific recombination. A trait that can be desired is altered development. A trait that can be desired is altered morphology (such as size and pigmentation). A trait that can be desired is an increase in yield. A trait that can be desired is increase in grain quality. A trait that can be desired is altered nutrient content. A trait that can be desired is altered starch quality. A trait that can be desired is altered starch quantity. A trait that can be desired is nitrogen fixation and/or utilization. A trait that can be desired is altered oil content and/or composition. A trait that can be desired is improved crop yield. A trait that can be desired is improved desirability of crops. A trait that can be desired is resistance to abiotic stress, such as drought, nitrogen, temperature, salinity, toxic metals or trace elements. A trait that can be desired is resistance to toxins such as pesticides and herbicides. A trait that can be desired is resistance to biotic stress, such as attacks by fungi, viruses, bacteria, insects, and nematodes, and development of diseases associated with these organisms.

Determining the trait of interest can be conducted by a number of different means. Accordingly, the trait of interest can be determined by any method known in the art. It will be appreciated by the skilled person that method of determination will be dependent on the characteristics of the trait of interest.

For example, a plant with a trait of interest can be selected by physical inspection when said trait of interest has a visible attribute such as flower colour, fruit size and fruit shape.

As used herein the term “phenotypic assay” includes any test that is used to select a particular plant or sub-group of plants that exhibit a trait of interest.

Alternatively, the trait of interest can be determined by “genotyping”, which is defined herein as the process of determining differences in the genotype of an individual by examining the DNA sequence using biological assays and comparing it to a reference sequence (e.g, a control or wild-type plant sequence).

Current methods of genotyping include for example, restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, and hybridization to DNA microarrays or beads. Furthermore, whole genome sequencing can also be used.

In alternative instances the trait of interest may only become apparent once the plant is subjected to transcriptomic or metabolomic analysis of the plant.

As used herein “transcriptomic analysis” is defined as a technique to study the sum of ail of a plant's RNA transcripts. A transcriptome captures a snapshot in time of the total transcripts present in a cell. Non limiting examples to determine the transcriptome of a plant include RNA-sequencing and microarrays.

As used herein “metabolomic analysis” is used herein to refer to the study of small-molecule metabolite profiles. Techniques known in the art for determining metabolite profiles are gas chromatography mass spectrometry (GC-MS), liquid chromatography mass spectrometry (LC-MS), high performance liquid chromatography (HPLC), capillary electrophoresis (CE) and nuclear magnetic resonance (NMR).

The plant methods described herein can include additional steps in which the modified plant is either grown or grown to seed. These additional steps would be known to a skilled person. The purpose of growing the resulting plant or growing the plant to seed can be used to assist in characterising the plant in order to determine if the plant, or progeny thereof, has the desired trait.

As the tandem duplication events have been observed to occur at random throughout the genome, a plurality of plants subjected to the methods described herein will have at least one tandem duplication located at different locations within the plant genome. Accordingly, the resulting plants can be screened for one or more traits of interest. Therefore, the method may comprise screening a population of plants. As used herein, “population of plants” refers to a plurality of plants each having reduced or abolished expression of at least one TONSOKU nucleic acid sequence and/or reduced or abolished level of a TONSOKU polypeptide and/or reduced or abolished activity of a TONSOKU polypeptide in the plant and increased endogenous tandem duplication events.

As such the methods described herein can be used to generate alternative plant lines to the T-DNA insertion lines that are widely used in plant genomic engineering (Jupe et al., 2019). Examples of Arabidopsis thaliana T-DNA insertion plant collections are SALK, SAIL and WISC. Whilst these plant lines are used routinely by plant geneticists adverse effects can be associated with inserting foreign gene-fragments which lead to unanticipated genomic changes.

In contrast, the methods described herein are not associated with the above difficulties because they utilise an endogenous process to increase the levels of tandem duplications in the plants. In other words the methods described herein increase the copy number of at least one endogenous (e.g. naturally occurring) gene.

The methods described herein can be employed in breeding programmes, for example in breeding programmes for an agronomically important plant species. As used herein, “breeding” is the genetic manipulation of living organisms.

The methods described herein may further comprise identifying a plant with a trait of interest.

The aspects of the invention involve recombination DNA technology and exclude embodiments that are solely based on generating plants by traditional breeding methods. Aspects of the invention are demonstrated by the following non-limiting examples.

### EXAMPLES

### Example 1: Methods for Generating Tonsoku⁻ A. thaliana and C. elegans and Results

To assess tandem duplication events in plants deficient for TONSOKU/BRUSHY1/MGOUN3 the inventors ordered Arabidopsis thaliana seeds (SAIL 525_A01, Col-0 background) and identified 5 plants homozygous for a T-DNA insertion into TONSOKU/BRUSHY1/MGOUN3. From these, 5 homozygous plant seeds were collected and grown 20 F1 plants after which genomic DNA was isolated from the flowers. A total of 50-200 ng of DNA was used as input for TruSeq Nano LT library preparation (Illumina), which was performed on an automated liquid handling platform (Beckman Coulter). DNA was sheared using sonication (Covaris) to average fragment lengths of 450 nt. Barcoded libraries were sequenced as pools on Novaseq 6000 S4 Reagent Kit generating 2×151 read pairs using standard settings (Illumina). BCL output from the HiSeqX and Novaseq6000 platform was converted using bcl2fastq tool (Illumina, versions 2.20 has been used) using default parameters. To detect genomic changes in the background of these TONSOKU-deficient plants we performed mapping via BWA-MEM after which duplicate reads were marked. Pindel (a tool designed to detect structural variations from paired-end sequencing data) was used to detect copy-number variations within each sample (Ye at al., 2009 Bioinformatics). Tandem duplication events were considered as real events if they were observed ≥5 times and manual inspection of the genomic location confirmed increased coverage over the reported location. Only events uniquely reported in one of the samples were considered to exclude mutations prior to homozygosity of the TONSOKU/BRUSHY1/MGOUN3 mutation. The results are shown in FIGS. 10 & 1D.

To assess tandem duplication events in C. elegans animals deficient for tnsl-1/K02B12.5 the inventors targeted tnsl-1 via CRISPR/Cas9 and identified 1 animal heterozygous for a deletion in tnsl-1, causing a frame shift, which results in a severely truncated protein. Homozygous animals were obtained in the subsequent generation. 10 clonal sub-populations were grown for 50 generations after which genomic DNA was isolated from a single animal. A total of 50-200 ng of DNA was used as input for TruSeq Nano LT library preparation (Illumina), which was performed on an automated liquid handling platform (Beckman Coulter). DNA was sheared using sonication (Covaris) to average fragment lengths of 450 nt. Barcoded libraries were sequenced as pools on Novaseq 6000 S4 Reagent Kit generating 2×151 read pairs using standard settings (Illumina). BCL output from the HiSeqX and Novaseq6000 platform was converted using bcl2fastq tool (Illumina, versions 2.20 has been used) using default parameters. To detect genomic changes in the background of these TONSOKU-deficient animals we performed mapping via BWA-MEM after which duplicate reads were marked. Pindel (a tool designed to detect structural variations from paired-end sequencing data) was used to detect copy-number variations within each sample (Ye at al., 2009 Bioinformatics). Tandem duplication events were considered as real events if they were observed times and manual inspection of the genomic location confirmed increased coverage over the reported location. Only events uniquely reported in one of the samples were considered to exclude mutations prior to homozygosity of the tnsl-1 mutation.

### Example 2: Generation of TONSOKU-Deficient Tomato Plants

The present example will demonstrate an increasing endogenous genome modification in a crop plant, namely tomato (Solanum lycopersum). The TONSOKU gene from tomato was identified from the NCBI database (release 103) as accession no RefSeq XM_019211119.2 and RefSeq XM_019211120.2 based on a BLAST search using the TONSOKU sequence.

TONSOKU-deficient tomato mutants are created by targeting the TONSOKU using CRISPR and self-pollinating to create homozygous mutants in the next generation. Briefly, a T-DNA construct is prepared encoding a kanamycin-selectable marker, a Cas9 enzyme (plant codon-optimized Cas9-pcoCas9 (Li et al. 2013 Nat Biotechnol 31:688-691)) and guide RNA, directing the Cas9 enzyme to the TONSOKU locus. The expression of Cas9 is under control of the 35S promoter and the guide RNA is under control of the U3 (AtU3) promoter. Tomato cotyledon explants are transformed by immersion in Agrobacterium suspension, selected for kanamycin resistance, and screened for TONSOKU mutations. Plantlets are screened for TONSOKU mutations using the Surveyor assay (Voytas 2013 Annu Rev Plant Biol 64:327-350) and plantlets containing an inactivating mutation in TONSOKU are grown and self-pollinated to create homozygous mutants in the next generation.

The effect of TONSOKU on endogenous genome modification is demonstrated using WGS performed on wild-type tomato plants and on TONSOKU-deficient tomato mutants, as already described for C. elegans and A. thaliana.

### Example 3: Generation of TONSOKU-Deficient Crop Plants

A crop plant, e.g. wheat, soybean, rice, cotton, corn or brassica plant having a mutation in one or more TONSOKU genes (e.g. in one or more homologous genes) is identified or generated via (random) mutagenesis or targeted knockout (e.g. using a sequence specific nuclease such as a meganuclease, a zinc finger nuclease, a TALEN, Crispr/Cas9, Crispr/Cpf1 etc). Reduction in TONSOKU expression and/or activity is confirmed by Q-PCR, western blotting or the like.

A crop plant, e.g. wheat, soybean, rice, cotton or brassica plant, is transformed with a construct encoding a TONSOKU inhibitory nucleic acid molecule or TONSOKU binding molecule (e.g. encoding a TONSOKU hairpin RNA, antibody, etc, under control of a constitutive or inducible promoter). Reduction in TONSOKU expression and/or activity is confirmed by Q-PCR, western blotting or the like.

### Example 4: Tandem Tandem-Duplication Formation in Arabidopsis thaliana with a Homozygous Mutation in TONSOKU

Arabidopsis thaliana plants with a homozygous mutation in the gene TONSOKU were grown and whole-genome sequenced to determine genomic alterations. An experiment was performed as follows: seeds were taken from a single plant (P0) with a homozygous mutation in TONSOKU and from these seeds 10 plants were grown (F1 generation). For this experiment we made use of the plant line with name/stock number: SAIL_525_A01/CS822237, which has a T-DNA insertion in the middle of the TONSOKU gene (gene number AT3G18730). These 10 sublines were grown in parallel for a total of three generations to allow them to accumulate de novo mutations during unperturbed growth. In the F3 generation DNA was isolated from flower buds and whole-genome sequenced as well as DNA from a pool of PO plant. To detect copy number variations in the sequenced plants three structural-variant callers were used: Pindel, Gridss and Manta. To determine the frequency of genome alterations for each subline only genomic alterations that were unique for that subline were considered (i.e. mutations that are not present in the PO sample and not present in any other F3 sample).

The frequency of de novo tandem duplications events in plants deficient for TONSOKU was found to be 7.0±1.5 per generation (FIG. 3). The median size of the tandem duplications is 199,589 (the 25-75 percentile range is 139,689-343,006) base-pair. The tandem duplications appear to be randomly distributed over the genome of Arabidopsis thaliana. In previous experiments we have not detected any tandem duplication in this size range in TONSOKU proficient plants lines that were grown for in total 80 generations.

During the propagation of the 10 mutation accumulation lines, the F1, F2 and F3 progeny plants were all inspected for novel phenotypic characteristics (e.g. plant morphology, rosette size and flowering time). One F3 population was identified in which ˜75% of the plants displayed early flowering, indicative of segregation of a dominant de novo generated trait, and one population in which the majority of the plants displayed late flowering. In addition, F3 individuals with novel rosette and inflorescence phenotypes were observed. Together, these observations provide proof of principle that novel inheritable traits can be obtained by reducing Tonsoku expression.

### REFERENCES

Shin Takeda, Zerihun Tadele, Ingo Hofmann, Aline V. Probst, Karel J. Angelis, Hidetaka Kaya, Takashi Araki, Tesfaye Mengiste, Ortrun Mittelsten Scheid, Kei-ichi Shibahara, Dierk Scheel, and Jerzy Paszkowski; BRU1, a novel link between responses to DNA damage and epigenetic gene silencing in Arabidopsis, Genes Dev. 2004 Apr. 1; 18(7): 782-793
Jupe F, Rivkin A C, Michael T P, Zander M, Motley S T, et al. (2019) The complex architecture and epigenomic impact of plant T-DNA insertions. PLOS Genetics 15(1): e1007819
Yusuke Ohno, Jarunya Narangajavana, Akiko Yamamoto, Tsukaho Hattori, Yasuaki Kagaya, Jerzy Paszkowski, Wilhelm Gruissem, Lars Hennig and Shin Takeda; Ectopic Gene Expression and Organogenesis in Arabidopsis Mutants Missing BRU1 Required for Genome Maintenance, GENETICS Sep. 1, 2011 vol. 189 no. 1 83-95
Burrage L C, Reynolds J J, Baratang N V, Phillips J B, Wegner J, McFarquhar A, Higgs M R, Christiansen A E, Lanza D G, Seavitt J R, Jain M, Li X, Parry D A, Raman V, Chitayat D, Chinn I K, Bertuch A A, Karaviti L, Schlesinger A E, Earl D, Bamshad M, Savarirayan R, Doddapaneni H, Muzny D, Jhangiani S N, Eng C M, Gibbs R A, Bi W, Emrick L, Rosenfeld J A, Postlethwait J, Westerfield M, Dickinson M E, Beaudet A L, Ranza E, Huber C, Cormier-Daire V, Shen W, Mao R, Heaney J D, Orange J S; University of Washington Center for Mendelian Genomics; Undiagnosed Diseases Network, Bertola D, Yamamoto G L, Baratela W A R, Butler M G, Ali A, Adeli M, Cohn D H, Krakow D, Jackson A P, Lees M, Offiah A C, Carlston C M, Carey J C, Stewart G S, Bacino C A, Campeau P M, Lee B; Bi-allelic Variants in TONSL Cause SPONASTRIME Dysplasia and a Spectrum of Skeletal Dysplasia Phenotypes. Am J Hum Genet. 2019 Mar. 7; 104(3):422-438
O'Donnell L, Panier S, Wildenhain J, Tkach J M, Al-Hakim A, Landry M C, Escribano-Diaz C, Szilard R K, Young J T, Munro M, Canny M D, Kolas N K, Zhang W, Harding S M, Ylanko J, Mendez M, Mullin M, Sun T, Habermann B, Datti A, Bristow R G, Gingras A C, Tyers M D, Brown G W, Durocher D. The MMS22L-TONSL complex mediates recovery from replication stress and homologous recombination; Mol Cell. 2010 Nov. 24; 40(4):619-31
Wang, Y., Xiong, G., Hu, J. et al. Copy number variation at the GL7 locus contributes to grain size diversity in rice. Nat Genet 47, 944-948 (2015)
Sambrook, et al., (1989) Molecular Cloning: A Library Manual (2d ed., Cold Spring Harbor Laboratory Press, Plainview, N.Y.
Walker and Gaastra, eds. (1983) Techniques in Molecular Biology (MacMillan Publishing Company, New York)
Soazig Guyomarc'h, Moussa Benhamed, Gaëtan Lemonnier, Jean-Pierre Renou, Dao-Xiuz Zhou, Marianne Delarue, MGOUN3: evidence for chromatin-mediated regulation of FLC expression, Journal of Experimental Botany, Volume 57, Issue 9, June 2006, Pages 2111-2119
Soazig Guyomarc'h, Teva Vernoux, Jan Traas, Dao-Xiu Zhou, Marianne Delarue, MGOUN3, an Arabidopsis gene with Tetratrico Peptide-Repeat-related motifs, regulates meristem cellular organization, Journal of Experimental Botany, Volume 55, Issue 397, 1 Mar. 2004, Pages 673-684
Suzuki, T., Inagaki, S., Nakajima, S., Akashi, T., Ohto, M.-a., Kobayashi, M., Seki, M., Shinozaki, K., Kato, T., Tabata, S., Nakamura, K. and Morikami, A. (2004), A novel Arabidopsis gene TONSOKU is required for proper cell arrangement in root and shoot apical meristems. The Plant Journal, 38: 673-684
Li J F, Norville J E, Aach J, et al. Multiplex and homologous recombination-mediated genome editing in Arabidopsis and Nicotiana benthamiana using guide RNA and Cas9. Nat Biotechnol.
Kunkel T A. Rapid and efficient site-specific mutagenesis without phenotypic selection. Proc Natl Acad Sci USA. 1985; 82(2):488-492
Kunkel T A, Roberts J D, Zakour R A. Rapid and efficient site-specific mutagenesis without phenotypic selection. Methods Enzymol. 1987; 154:367-82
Patrick J. Krysan, Jeffery C. Young, Michael R. Sussman; The Plant Cell, December 1999, 11 (12) 2283-2290
Saredi, G., Huang, H., Hammond, C. et al. H4K20me0 marks post-replicative chromatin and recruits the TONSL-MMS22L DNA repair complex. Nature 534, 714-718 (2016)
Henikoff S, Till B J, Comai L. TILLING. Traditional mutagenesis meets functional genomics. Plant Physiol. 2004; 135(2):630-636
Cermak T, Doyle E L, Christian M, et al. Efficient design and assembly of custom TALEN and other TAL effector-based constructs for DNA targeting [published correction appears in Nucleic Acids Res. 2011 Sep. 1; 39(17):7879]. Nucleic Acids Res. 2011; 39(12):e82
Mishell et al., Prevention of the immunosuppressive effects of glucocorticosteroids by cell-free factors from adjuvant-activated accessory cells. 1980 Immunopharmacology, ISSN: 0162-3109, Vol: 2, Issue: 3, Page: 233-45
Schirrmann T, Meyer T, Schutte M, Frenzel A, Hust M. Phage display for the generation of antibodies for proteome research, diagnostics and therapy. Molecules. 2011; 16(1):412-426
Daniel F. Voytas, Plant Genome Engineering with Sequence-Specific Nucleases, Annual Review of Plant Biology 2013 64:1, 327-350
Khatodia et al. Frontiers in Plant Science 2016 7: article 506
Ye K, Schulz M H, Long Q, Apweiler R, Ning Z. Pindel: a pattern growth approach to detect break points of large deletions and medium sized insertions from paired-end short reads. Bioinformatics. 2009; 25(21):2865-2871
U.S. Pat. Nos. 4,873,192; 8,440,431, 8,440,432; 8,450,471; 8,697,359 and 6,635,805

SEQUENCES

A. thaliana TONSOKU amino acid sequence [SEQ ID NO: 1]

| | |
|---|---|
| 1 | mgrldvaaak rayrkaeevg drreqarwan nvgdilknhg eyvdalkwfr idydisvkyl |
| 61 | pgkdllptcq slgeiylrle nfeealiyqk khlqlaeean dtvekqract qlgrtyhemf |
| 121 | lkseddceai qsakkyfkka melaqilkek pppgessgfl eeyinahnni gmldldldnp |
| 181 | eaartilkkg lqicdeeevr eydaarsrlh hnlgnvfmal rswdeakkhi emdinichki |
| 241 | nhvqgeakgy inlaelhnkt qkyidallcy gkasslaksm qdesalveqi ehntkivkks |
| 301 | mkvmeelree elmlkklsae mtdakgtsee rksmlqvnac lgslidkssm vfawlkhlqy |
| 361 | skrkkkisde lcdkeklsda fmivgesyqn lrnfrkslkw firsyeghea ignlegqala |
| 421 | kinigngldc igewtgalqa yeegyrialk anlpsiqlsa ledihyihmm rfgnaqkase |
| 481 | lketiqnlke sehaekaecs tqdecsetds eghanvsndr pnacsspqtp nslrserlad |
| 541 | ldeanddvpl isflqpgkrl fkrkqvsgkq dadtdqtkkd fsvvadsqqt vagrkrirvi |
| 601 | lsddesetey elgcpkdssh kvlrqneevs eesmyfdgai nytdnraiqd nveegscsyt |
| 661 | plhpikvapn vsncrslsnn iavettgrrk kgsqcdvgds ngtscktgaa lvnfhayskt |
| 721 | edrkikieie nehialdscs hddesvkvel tclyylqlpd dekskgllpi ihhleyggrv |
| 781 | lkplelyail rdssenvvie asvdgwvhkr lmklymdccq slsekpsmkl lkklyiseve |
| 841 | ddinvsecel qdisaapllc alhvhniaml dlshnmlgng tmeklkqlfa sssqmygalt |
| 901 | ldlhcnrfgp talfqicecp vlftrlevln vsrnrltdac gsylstivkn cralysinve |
| 961 | hcsltsrtiq kvanaldsks glsqlcigyn npvsgssiqn llaklatlss faelsmngik |
| 1021 | lssqvvdsly alvktpslsk llvgssgigt dgaikvtesl cyqkeetvkl dlsccglass |
| 1081 | ffiklnqdvt ltssilefnv ggnpiteegi salgellrnp csnikvlils kchlklagll |
| 1141 | ciiqalsdnk nleelnlsdn akiedetvfg qpvkersvmv eqehgtcksv tsmdkeqelc |
| 1201 | etnmecddle vadsedeqie egtatsssls lprknhivke lstalsmanq lkildlsnng |
| 1261 | fsvealetly mswsssssrt giaqrhvkee tvhfyvegkm ccgvksccrk d |

A. thaliana TONSOKU promoter sequence [SEQ ID NO: 2]

| | |
|---|---|
| 1 | cctggaaaac cgatgtcaca gtcgatcatc tcatccattc gcaactgaat cagaactcaa |
| 61 | gaagtcatca taacgaagca aagccacaga aacaagagga gactgttttt catgatactt |
| 121 | gtgagttggt tagtcactcg tgtaactcag attgcccacg atcagatgag gaagataagc |
| 181 | aatgcgtcga tgccaccaaa ggagaagaca agagctccat tcaagaagta gaagaagcaa |
| 241 | ccgaaccagt aagtttggag gaagaagaaa ggttaagaca agagctggag gagatagaag |
| 301 | ctaagtatca ggaagatatg aaagagatag caacgaaaag agaagaggcc attatggaga |
| 361 | cgaagaaaaa gttgtctctg atgaagttaa agtaatagcc aaaaaagctc aaagaaaacg |
| 421 | ttgatactga tgaagagctt ttgtgttttt aatctctttt gtttaatttg ttggttggag |
| 481 | gagaagtgta gaaagatgaa gggtttctat ttgattaatt gagatttaat ttggttggtt |
| 541 | gttacaagtt agaacataaa aaatggttcc tgttaaaatg ttctaagaga ttgtccatta |
| 601 | tatatgattt tgtataaatt gaacatgtaa ttagttaata gccaactatt gtaataaaag |
| 661 | taatcaagcc ttttcgtgta aggaatcaat caacagagac gaaaatgtag taattaatta |
| 721 | taaccattaa gaggaagtcg ggaaaccaaa gaaatctaac attaagtctt tgaagaacac |
| 781 | aaagcataat caagcataga gaacaacatg gcaaaatcat caaaatcaga atcactgatc |
| 841 | tccaggaagt gtcttgatga tgtcggaatc accaggatca acgatgctga ggcaagaaac |
| 901 | tcggaagtat ttaccacaag cagtacccaa atcaacattg ttgccattgt agcgatgaac |
| 961 | tccaacttta gcaagcatcg catagtattc aatctctgac cttctcaacg gtgggcaatt |
| 1021 | gctagatatc aatatcagct tacctaaacc ccccaacaat atccaacaat tattcaacta |
| 1081 | aattacgagg aagacgaaca ctataatcaa tcgatgaaga gggattttaa atttttacct |
| 1141 | ttggagctgc gaagggattt gagaacagac ttgtatccaa gagtgtactt tccactcttc |
| 1201 | atcacaagag ctaatctgct gttgattcct tcatgggact tcttcgcctt cttctccgca |
| 1261 | accatttttc accgccggga agattcagat cgcaggttta caagagagag ttcttcttcg |
| 1321 | ggttcgggcg gcgcaaaatg atagtttata tagcgagtgc cttagaaccc ttagggtttt |
| 1381 | tttgttttct tgtcaggaga caggaggata taagaagccc aaaataaact cgacccaagg |
| 1441 | cccaaactaa aaggcctata acttcaggat ttagggtatg aaaatttcta atttaccctt |

A. thaliana TONSOKU genomic sequence [SEQ ID NO: 3]
GAATTTTGGCGGGATAGTTTGGGATGGGACCAAAAATTTGGCGACTGGAGAAAATGAGAAAATCAAAATC
ACTGAGAAAGAAATTTCGAGAAATCTGAAAATCGGAAGGAAGAAAACAAAAACCTTTCAATTGAAGAACG
GAGAAATCATCATCCGATGGGTCGATTAGATGTAGCTGCGGCGAAGAGAGCGTACCGGAAAGCAGAAGAA
GTGGGTGACCGGAGAGAACAGGCGAGGTGGGCTAACAATGTCGGCGATATCCTTAAGAATCATGGAGAGT
ACGTTGATGCTCTCAAGTGGTTTAGGATTGATTACGATATCTCCGTCAAGTATTTACCTGGGAAAGATTT
GTTACCTACTTGTCAGTCTCTTGGCGAGATCTATCTCCGCCTCGAAAATTTCGAAGAAGCCTTGATTTAT
CAGGTAAGCCCTCTTGAATCAATTGCTTTTTCCTACTTGGTTATTGTTGGCTTCCTGAATTTTCCGTGAA
TAATTTTGGTGTTTGAGTTTTTCATTTTGAATTTGTGTTTTTTTCTGGTGGTTGCAGAAGAAGCATTTAC
AGGTAGCTGAAGAAGCTAATGACACTGTGGAGAAGCAAAGAGCATGTACTCAACTTGGACGTACTTACCA
TGAAATGTTCTTGAAGTCTGAGGATGATTGTGAAGCCATTCAGAGTGCTAAAAAGTACTTTAAGAAAGCC
ATGGAACTTGCACAGATTCTCAAGGAGAAACCACCTCCTGGAGAATCTAGCGGATTCCTTGAGGAGTATA
TTAACGCACATAACAACATCGGTATGCTTGACCTTGATCTTGATAATCCTGAAGCAGCCCGTACTATTCT
TAAGAAAGGGCTGCAGATTTGCGATGAAGAGGAGGTGAGAGAGTATGATGCTGCTCGGAGTAGGCTTCAT
CATAACCTTGGAAACGTTTTTATGGCGCTGAGAAGTTGGGATGAAGCAAAGAAACACATTGAGATGGATA
TTAATATCTGTCATAAGATTAATCATGTCCAAGGAGAAGCGAAGGGGTATATCAATCTCGCTGAATTACA
CAACAAGACCCAAAAGTACATTGATGCTCTTTTATGTTATGGTAAAGCTTCTAGTCTAGCGAAATCTATG
CAAGACGAGAGTGCATTGGTTGAACAGATAGAGCATAATACCAAGATAGTCAAGAAATCCATGAAAGTTA
TGGAAGAATTGAGAGAAGAAGAGCTTATGCTTAAGAAGTTGTCTGCAGAAATGACTGATGCCAAAGGCAC
TTCGGAGGAACGAAAGTCTATGCTCCAAGTAAATGCTTGTCTTGGAAGTCTTATTGATAAATCTAGCATG
GTATTCGCATGGCTGAAGGTGAGTTTTATAACTTAAACACTCCTTCCTTTTTAGTCCTATCACTCCACCC
CATGTTCGCATTTATTTGAAAAGTTTCCAGAAGTTAAAGTTGTCCATCGTAGGGGTTTTTAATGATGAAT
AAGCATTGTGAGATTTCATCAGGTAGTATGGAGTAGGAAAAATATGCTATTTTCTTAGATTTGATTTAAG
TTTTGTGAACTTCTGCTATTGACACTGTCTTTTCAGATCAGTCAGGAAGACTATATTATCAAAGAATTAC
ATGATTCTTGTTCTCTCAAGAAAACCTATCTTTTGAATGCTGGGATAATATCTTTGTTCTGAACTTGCAA
AGTAAAGTTATTATGTGGCAAAACGATGATTATTCTGTATCATACGGATACTGAGTGATCCAAGTCTCTG
CATCACTGTTTCAATGACTTGTGATATAGTTTTGAAAGTTAAGTAGGAGGCTGCCATTTGAAGTTTGCAT
GCAACTAAAGGGTTGCTATTTCTTCTTTGAATGTCTTAGCATCTTCAATATTCAAAAAGGAAGAAGAAAA
TATCAGATGAACTCTGTGACAAGGAAAAGCTGAGTGATGCCTTCATGATTGTTGGAGAATCTTACCAAAA
TCTCAGAAATTTCAGAAAGTCCCTGAAGTGGTTCATAAGAAGTTATGAGGGACATGAAGCAATTGGTAAT
CTGGAGGTGAGATTTGTTTGCTTGCACGATTAATTATAAAAACCTATGTTCACTACTGTCATCAGAATTT
GATTCACAAAACCAGAAATAATTCATTAGGCCTCTACTGAACATTTTCTGTGGAAAACTGATTATACCTT
TTCTTGGATTTGTCAATATTATAGCTATTCTTCTTTCCTGATTCTAATATTCACTTATGGTGGTCTCTTG
TAGGGTCAAGCACTAGCGAAGATTAATATTGGTAATGGTTTGGACTGTATTGGGGAATGGACAGGAGCAC
TTCAGGCATATGAAGAGGGGTACAGGTAGATCCAATTATAAGTAATCTTTATCAAACTGCGCATTTGAGC
TATTATTTGGTTATGTTTGTGATTCAGTCCTAGTAAATCTACTTATTAATTTTCCTTGAGAGAACTGATA
ATTCCATTGAACAATATGACGGCGATGAAACTCATTTTTTTCTTAAAATGGAAAGAACACTTGAAGCAGA
GCAAATGTGAATGTGCTATAAAGTACTTAACTGCTTGTTGGTTGTCCCTTTCGACTAAGTTCACGAATTA
CTGCACTATGGCTTCTGAATAAATAATACAATGTACTTTGAATCAGTACTTCTCATGATAGTGGATAATT
ATAGCACATTTTGCATTTTCAATCACTTAAAATATTTTTTCTGTGACTTTCTTCTGCTATATTCAAACAC
ATCGCATATACATTTACGTGAATTTATACACACATACTGCATGCTAATAAATTAACTATTGGTCTTTCTG
GATTTATTTTCATTTGATCCTGCAGAATTGCTTTGAAAGCTAATCTTCCTTCAATCCAGCTTTCTGCACT
GGAAGATATACACTATATCCATATGATGAGATTTGGGAATGCTCAAAAAGCCAGGTAACAATTACTGTTT
TGTCACTGGACGGAATATGGATAGACACCAAATCTGGTGTAAGGTTTGCAGTTTCAAGTATTTCATTTTA
CTCATATATTATTTCTACTGTCTAGTGAATTGAAGGAAACAATACAAAATCTGAAGGAGTCAGAACATGC
TGAGAAAGCCGAATGTAGTACACAAGATGAATGCTCTGAAACTGACTCAGAAGGGCATGCGAATGTATCG
AATGATAGGCCAAATGCATGTAGCTCACCGCAAACACCAAATTCACTTAGATCAGAACGGTTAGCAGATC
TGGATGAAGCAAATGATGATGTGCCACTAATTTCATTTCTCCAGCCTGGAAAACGTCTGTTCAAAAGGAA
ACAAGTTTCAGGAAAACAAGATGCTGACACTGATCAGACGAAGAAAGATTTCTCTGTAGTAGCAGACTCT
CAGCAGACAGTTGCTGGTCGAAAGCGTATTCGAGTAATCCTCTCTGATGATGAAAGTGAGACCGAATATG
AGCTGGGATGCCCTAAAGACAGTTCTCACAAAGTTCTAAGGCAGAATGAAGAGGTTTCTGAGGAAAGTAT
GTATTTTGATGGTGCTATTAATTATACGGATAATCGTGCCATCCAAGATAATGTAGAAGAAGGTTCTTGC
TCGTATACGCCTCTCCATCCTATTAAGGTGGCTCCAAATGTCAGCAATTGTAGATCTTTGAGTAATAATA
TAGCTGTTGAAACAACTGGTCGTCGTAAAAAAGGATCTCAATGTGATGTTGGCGACTCCAACGGCACGTC
CTGCAAAACTGGAGCTGCTCTCGTGAACTTCCACGCTTACTCAAAAACTGAGGATGTGAGCAACTGTGAT
CTGGTTTTTGAGTTATCATTGACCATTCTTGGGATTGGATTTCATTTATTTTTCTACTTCGTCCAATCTT
CTTCATGATAACTATATGTTTTACTTGTTGCAGCGAAAAATAAAAATTGAAATTGAAAATGAACACATAG
CTTTAGACTCCTGTTCTCACGATGATGAGTCTGTGAAGGTGGAACTTACTTGCCTATACTATTTACAGCT
TCCTGACGATGAGAAATCTAAAGGTATGTGCTTTTGTTTTCTTAGCAAAACTTTAGGATGATCCCAGTTC
GGATCAGTCTCTATAATGCATGATCCCAGTTCGGATCAGTCCTATAATTCTCATCTCACGCTTAATAACA
TTTCTTTTGCTTTTTGATATCATTCCCCTTGTTTCCTAGCACGTTTTAAGTTTTGCTCTAAAAGTTTGAA
TCTTTGAACATTCAATTTGCGTTAGGTCTGTTGCCGATCATTCATCATTTGGAATATGGTGGAAGAGTTC
TGAAACCATTGGAACTATATGCGATTCTCAGGGACTCTTCTGAAAATGTTGTTATTGAAGCTTCCGTTGA
TGGTAAGTATTTCCTTGATAGAATTGGAATCTACTCATGATATTTGGATGTATGATTGTCAAGCTGATCA
TTCTATAAATTTGTTTTCATCACAAATTGTTCTCTCACTTTTTACATGATTGTGCTGAACCGCTGTATTG
GCTTTTAAGATTATGGTCATTGATTCTTCCCTCTTATTTATACACCACGGCTGAATCAGCATGAAATTAA
TTTGTTTTCAGGCTGGGTTCACAAGCGCCTGATGAAACTATACATGGACTGTTGCCAGTCGTTGTCAGAG
AAACCCAGTATGAAATTGCTTAAGAAATTATATATTTCGGAGGTGAGAGTATTACCCCAAATTTTAGCGG
TTAATGTATGAAATATTTTCTTCTCTTTGTTTGCTTTTCAACCTACTTAAAGCTAGCTAGTTACAAATTC
TTACTTTATTTGATGTATAATCTGAATGGTTATTTCGTTGTATGTTTATCAGGTAGAAGATGATATCAAT
GTGTCAGAATGTGAACTGCAAGACATATCAGCTGCTCCATTATTGTGTGCCCTCCATGTCCACAATATTG
CTATGTTGGATCTCTCCCACAATATGCTAGGTGAAAGTTGCCTCTGACGTCTTACTTAATTTAATGAGCT
GACCTAAGTGAGTTAGTTGGTTATGCATAGGGAACTACTAGGAAATTCAGAAGTGTTAATTTCCATCGTC
TCATTGGTTGTTAGGGAATGGAACAATGGAGAAATTGAAACAACTTTTTGCCTCATCAAGCCAGATGTAT
GGTGCTTTAACTTTGGATTTGCACTGCAATCGATTTGGTCCAACTGCTTTGTTTCAGGTACACTACTAGG
CCCAAAGCTAGAAAATTTCACATATTCATGTTATTTTCGTATTATTTAATATACTCCTCTTTACCAGATC
TGTGAATGCCCTGTTCTGTTCACTCGACTTGAAGTCCTCAATGTGTCCAGGAATCGACTTACAGATGCTT
GTGGATCATACCTCTCAACTATAGTGAAAAATTGCCGGGGTATAGATTTTTTTTTTTTTTTTTTTAAATT
ATGATAATTCATTTACAGTATCTAAATGCCCTGATGGTATGTTTTGTTTCTTGGTTTCACTGGTGTCTTA
TAAACCCAGTAGATAGATATATGAAATACCTGATATTAGGTTTAATAATCTTAAACATTTTCTTCCATTC
ACTAGCTTACATTAATGTGTCCCCTTTTGTTTCTTAGCACTTTACAGCTTGAATGTGGAACATTGTTCAC
TTACATCAAGAACAATCCAAAAGGTAGCTAATGCTTTGGATTCGAAGTCAGGACTTTCACAACTCTGTAT
AGGTGATCTTTCTAATTTGTTATGTACATTCAATTTATTTTTTTTATCTCGTTTCAGTTTGCTGAAGTTG
GTGGATCCGTATATGGCAGGTTATAATAATCCTGTTTCAGGGAGTAGTATTCAAAACCTCTTGGCTAAAT
TGGCTACTCTAAGCAGGTTGAAAGAAACACATTTTAAAGCTGTTTTTTTTTTATACGTAAATCCATCTAA
CATGATCATATGTCAAAACACTGCAGCTTTGCAGAACTGAGCATGAATGGCATAAAGCTGAGCAGCCAAG
TTGTTGATAGCCTTTATGCACTTGTTAAGACTCCATCTCTGTCAAAACTTTTGGTTGGCAGCAGTGGAAT
AGGAACGGTAATGATATGTTTAGCATTCAAAATTGAATTCTTATATTGTGATAAATACATCTTTTTTTAT
CTGACGATACTATACAAATTATTCTAGGACGGGGCTATAAAAGTTACTGAATCTCTATGTTATCAGAAGG
AAGAAACTGTGAAGCTCGACCTTTCATGTTGTGGACTAGCTTCCTCTTTCTTTATTAAGCTCAACCAAGA
TGTTACTCTAACCTCTAGCATTCTTGAGTTTAATGTTGGAGGAAATCCAATCACCGAAGAGGTATGTTTT
CTATGACTCAACATCCTAAAGCTCTTTTATCTAACTCTGTTGAGGCTGCAATGGTGATAGAATAAGCTAA
AGAATTTGCAATCATTCAACATGTGATTTTAAGTTCATGTCTTCTCAAAGCATAACTGACTCTCTGAAAC
ACTAAACAAACAGGGAATCAGTGCACTTGGGGAGCTGCTTAGGAATCCTTGTTCAAACATAAAAGTTCTT
ATTCTAAGCAAGTGTCATCTGAAGCTCGCTGGGCTTCTATGCATAATTCAAGCACTTTCAGGTCTGAAGT
ATTCTTGTAGCTGCTATTAAACAAAAGATCTTCTCCTTTTTAAACTATCAACTAAATGCTCTGCAGATAA
TAAGAATCTTGAAGAGCTTAATCTTTCTGACAATGCTAAGATAGAAGATGAGACTGTGTTTGGCCAACCT
GTGAAGGAAAGATCAGTAATGGTAGAGCAAGAACATGGAACATGTAAATCTGTCACCTCAATGGACAAAG
AACAAGAGCTATGTGAAACCAATATGGAGTGTGATGATCTCGAAGTTGCAGACAGCGAAGATGAACAAAT
AGAGGAAGGAACTGCAACCTCGAGTAGTCTTAGTTTGCCACGCAAGAACCATATCGTGAAAGAGCTTTCT
ACCGCTCTTTCAATGGCTAACCAGTTGAAGATTCTGGACTTAAGCAACAATGGGTTCTCAGTTGAAGCCT
TGGAAACATTATACATGTCATGGTCATCATCAAGCTCCCGAACTGGCATCGCCCAAAGGCATGTAAAAGA
AGAGACTGTCCATTTTTATGTCGAAGGAAAGATGTGTTGCGGAGTCAAATCATGCTGCAGAAAGGACTGA
AGAAGATCTTGTCTGAAACTGTATTTGCCAATAATAAACCTCTGTTTTTAAATATTGAGTATTTTTATTT
AGAGCGTTTGCAGAAATTTTTACATATTGATATTTACACATTTGGGTTGTGATGTGTAAATTTGCTGCAG
TTTAAGCGTTAATGCTCATATAAATTTAGTGACGTTAATCTTATGCAACTTTTTAAAAAATGTAAAAATT
A. thaliana TONSOKU cDNA sequence [SEQ ID NO: 4]

| | |
|---|---|
| 1 | gaattttggc gggatagttt gggatgggac caaaaatttg gcgactggag aaaatgagaa |
| 61 | aatcaaaatc actgagaaag aaatttcgag aaatctgaaa atcggaagga agaaaacaaa |
| 121 | aacctttcaa ttgaagaacg gagaaatcat catccgatgg gtcgattaga tgtagctgcg |
| 181 | gcgaagagag cgtaccggaa agcagaagaa gtgggtgacc ggagagaaca ggcgaggtgg |
| 241 | gctaacaatg tcggcgatat ccttaagaat catggagagt acgttgatgc tctcaagtgg |
| 301 | tttaggattg attacgatat ctccgtcaag tatttacctg ggaaagattt gttacctact |
| 361 | tgtcagtctc ttggcgagat ctatctccgc ctcgaaaatt tcgaagaagc cttgatttat |
| 421 | cagaagaagc atttacagct agctgaagaa gctaatgaca ctgtggagaa gcaaagagca |
| 481 | tgtactcaac ttggacgtac ttaccatgaa atgttcttga agtctgagga tgattgtgaa |
| 541 | gccattcaga gtgctaaaaa gtactttaag aaagccatgg aacttgcaca gattctcaag |
| 601 | gagaaaccac ctcctggaga atctagcgga ttccttgagg agtatattaa cgcacataac |
| 661 | aacatcggta tgcttgacct tgatcttgat aatcctgaag cagcccgtac tattcttaag |
| 721 | aaagggctgc agatttgcga tgaagaggag gtgagagagt atgatgctgc tcggagtagg |
| 781 | cttcatcata accttggaaa cgtttttatg gcgctgagaa gttgggatga agcaaagaaa |
| 841 | cacattgaga tggatattaa tatctgtcat aagattaatc atgtccaagg agaagcgaag |
| 901 | gggtatatca atctcgctga attacacaac aagacccaaa agtacattga tgctctttta |
| 961 | tgttatggta aagcttctag tctagcgaaa tctatgcaag acgagagtgc attggttgaa |
| 1021 | cagatagagc ataataccaa gatagtcaag aaatccatga aagttatgga agaattgaga |
| 1081 | gaagaagagc ttatgcttaa gaagttgtct gcagaaatga ctgatgccaa aggcacttcg |
| 1141 | gaggaacgaa agtctatgct ccaagtaaat gcttgtcttg gaagtcttat tgataaatct |
| 1201 | agcatggtat tcgcatggct gaagcatctt caatattcaa aaaggaagaa gaaaatatca |
| 1261 | gatgaactct gtgacaagga aaagctgagt gatgccttca tgattgttgg agaatcttac |
| 1321 | caaaatctca gaaatttcag aaagtccctg aagtggttca taagaagtta tgagggacat |
| 1381 | gaagcaattg gtaatctgga gggtcaagca ctagcgaaga ttaatattgg taatggtttg |
| 1441 | gactgtattg gggaatggac aggagcactt caggcatatg aagaggggta cagaattgct |
| 1501 | ttgaaagcta atcttccttc aatccagctt tctgcactgg aagatataca ctatatccat |
| 1561 | atgatgagat ttgggaatgc tcaaaaagcc agtgaattga aggaaacaat acaaaatctg |
| 1621 | aaggagtcag aacatgctga gaaagccgaa tgtagtacac aagatgaatg ctctgaaact |
| 1681 | gactcagaag ggcatgcgaa tgtatcgaat gataggccaa atgcatgtag ctcaccgcaa |
| 1741 | acaccaaatt cacttagatc agaacggtta gcagatctgg atgaagcaaa tgatgatgtg |
| 1801 | ccactaattt catttctcca gcctggaaaa cgtctgttca aaaggaaaca agtttcagga |
| 1861 | aaacaagatg ctgacactga tcagacgaag aaagatttct ctgtagtagc agactctcag |
| 1921 | cagacagttg ctggtcgaaa gcgtattcga gtaatcctct ctgatgatga aagtgagacc |
| 1981 | gaatatgagc tgggatgccc taaagacagt tctcacaaag ttctaaggca gaatgaagag |
| 2041 | gtttctgagg aaagtatgta ttttgatggt gctattaatt atacggataa tcgtgccatc |
| 2101 | caagataatg tagaagaagg ttcttgctcg tatacgcctc tccatcctat taaggtggct |
| 2161 | ccaaatgtca gcaattgtag atctttgagt aataatatag ctgttgaaac aactggtcgt |
| 2221 | cgtaaaaaag gatctcaatg tgatgttggc gactccaacg gcacgtcctg caaaactgga |
| 2281 | gctgctctcg tgaacttcca cgcttactca aaaactgagg atcgaaaaat aaaaattgaa |
| 2341 | attgaaaatg aacacatagc tttagactcc tgttctcacg atgatgagtc tgtgaaggtg |
| 2401 | gaacttactt gcctatacta tttacagctt cctgacgatg agaaatctaa aggtctgttg |
| 2461 | ccgatcattc atcatttgga atatggtgga agagttctga aaccattgga actatatgcg |
| 2521 | attctcaggg actcttctga aaatgttgtt attgaagctt ccgttgatgg ctgggttcac |
| 2581 | aagcgcctga tgaaactata catggactgt tgccagtcgt tgtcagagaa acccagtatg |
| 2641 | aaattgctta agaaattata tatttcggag gtagaagatg atatcaatgt gtcagaatgt |
| 2701 | gaactgcaag acatatcagc tgctccatta ttgtgtgccc tccatgtcca caatattgct |
| 2761 | atgttggatc tctcccacaa tatgctaggg aatggaacaa tggagaaatt gaaacaactt |
| 2821 | tttgcctcat caagccagat gtatggtgct ttaactttgg atttgcactg caatcgattt |
| 2881 | ggtccaactg ctttgtttca gatctgtgaa tgccctgttc tgttcactcg acttgaagtc |
| 2941 | ctcaatgtgt ccaggaatcg acttacagat gcttgtggat catacctctc aactatagtg |
| 3001 | aaaaattgcc gggcacttta cagcttgaat gtggaacatt gttcacttac atcaagaaca |
| 3061 | atccaaaagg tagctaatgc tttggattcg aagtcaggac tttcacaact ctgtataggt |
| 3121 | tataataatc ctgtttcagg gagtagtatt caaaacctct tggctaaatt ggctactcta |
| 3181 | agcagctttg cagaactgag catgaatggc ataaagctga gcagccaagt tgttgatagc |
| 3241 | ctttatgcac ttgttaagac tccatctctg tcaaaacttt tggttggcag cagtggaata |
| 3301 | ggaacggacg gggctataaa agttactgaa tctctatgtt atcagaagga agaaactgtg |
| 3361 | aagctcgacc tttcatgttg tggactagct tcctctttct ttattaagct caaccaagat |
| 3421 | gttactctaa cctctagcat tcttgagttt aatgttggag gaaatccaat caccgaagag |
| 3481 | ggaatcagtg cacttgggga gctgcttagg aatccttgtt caaacataaa agttcttatt |
| 3541 | ctaagcaagt gtcatctgaa gctcgctggg cttctatgca taattcaagc actttcagat |
| 3601 | aataagaatc ttgaagagct taatctttct gacaatgcta agatagaaga tgagactgtg |
| 3661 | tttggccaac ctgtgaagga aagatcagta atggtagagc aagaacatgg aacatgtaaa |
| 3721 | tctgtcacct caatggacaa agaacaagag ctatgtgaaa ccaatatgga gtgtgatgat |
| 3781 | ctcgaagttg cagacagcga agatgaacaa atagaggaag gaactgcaac ctcgagtagt |
| 3841 | cttagtttgc cacgcaagaa ccatatcgtg aaagagcttt ctaccgctct ttcaatggct |
| 3901 | aaccagttga agattctgga cttaagcaac aatgggttct cagttgaagc cttggaaaca |
| 3961 | ttatacatgt catggtcatc atcaagctcc cgaactggca tcgcccaaag gcatgtaaaa |
| 4021 | gaagagactg tccattttta tgtcgaagga aagatgtgtt gcggagtcaa atcatgctgc |
| 4081 | agaaaggact gaagaagatc ttgtctgaaa ctgtatttgc caataataaa cctctgtttt |
| 4141 | taaatattga gtatttttat ttagagcgtt tgcagaaatt tttacatatt gatatttaca |
| 4201 | catttgggtt gtgatgtgta aatttgctgc agtttaagcg ttaatgctca tataaattta |
| 4261 | gtgacgttaa tcttatgcaa ctttttaaaa aatgtaaaaa tt |

A single unit sequence [SEQ ID NO: 5]
ATTCG
A polynucleotide with two tandem repeats of the unit sequence [SEQ ID NO: 6]
ATTCGATTCG
A polynucleotide with three tandem repeats of the unit sequence [SEQ ID NO: 7]
ATTCGATTCGATTCG
A polynucleotide with four tandem repeats of the unit sequence [SEQ ID NO: 8]
ATTCGATTCGATTCGATTCG
A single unit sequence [SEQ ID NO: 9]
TATACAG

1. A method of increasing endogenous genome modification in a plant cell, the method comprising:
(i) reducing or abolishing the expression of at least one TONSOKU nucleic acid sequence and/or reducing or abolishing the level of a TONSOKU polypeptide and/or reducing or abolishing an activity of a TONSOKU polypeptide in the plant cell.

2. The method of claim 1, wherein the method increases endogenous insertions within the genome of the plant cell.

3. The method of claim 1 or 2, wherein the method results in at least one tandem duplication event occurring within the genome of the plant cell.

4. The method of claim 3, wherein the method results in at least two tandem duplication events occurring within the genome of the plant cell, and wherein the at least two tandem duplications occur at different locations within the genome.

5. The method of claim 4, wherein the method results in at least three tandem duplication events occurring within the genome of the plant cell, and wherein the at least three tandem duplication events occur at different locations within the genome.

6. The method of claims 3 to 5, wherein each tandem duplication event occurs at a random location within the genome of the plant cell.

7. The method of claims 3 to 6, wherein a unit sequence that is repeated by the tandem duplication event is 50-500 kilobases in size.

8. The method of any preceding claim, wherein the method comprises introducing at least one mutation into:
(i) the at least one TONSOKU gene;
(ii) an upstream promoter of the at least one TONSOKU gene; or
(iii) a regulatory element of the at least one TONSOKU gene.

9. The method of claim 8, wherein the mutation is a loss of function mutation.

10. The method of claim 8 or 9, wherein the mutation is an insertion, deletion or substitution.

11. The method of claims 8 to 10, wherein the mutation is introduced using a targeted genome modification technique.

12. The method of claim 11, wherein the targeted genome modification technique is selected from CRISPR/Cas9, ZFNs, TALENs or meganucleases.

13. The method of claims 8 to 12, wherein the mutation is introduced using mutagenesis.

14. The method of claim 13, wherein the mutagenesis is selected from: EMS, TILLING, transposon or T-DNA insertion.

15. The method of claims 8 to 14, wherein the plant cell is homozygous for the mutation.

16. The method of claims 1 to 7, wherein the method comprises using RNA interference to reduce or abolish the expression of the at least one TONSOKU nucleic acid sequence in the plant cell.

17. The method of any preceding claim, wherein the TONSOKU nucleic acid sequence comprises or consists of SEQ ID NO: 3 or 4.

18. The method of claims 1 to 7, wherein the method comprises using an inhibitor to reduce or abolish an activity of the TONSOKU polypeptide in the plant cell.

19. The method of any preceding claim, wherein the TONSOKU polypeptide comprises or consists of SEQ ID NO: 1.

20. The method of any preceding claim, wherein increasing endogenous genome modification in the plant cell is relative to a control plant cell or a wild-type plant cell.

21. The method of any preceding claim, wherein the plant cell is in a plant tissue, such as pollen, ovules, leaves, embryos, roots, root tips, anthers, flowers, fruits, stems shoots or seeds.

22. The method of any preceding claim, wherein the plant cell is in a plant part, such as pollen, ovules, leaves, embryos, roots, root tips, anthers, flowers, fruits, stems, shoots, scions, rootstocks, seeds, protoplasts or calli.

23. The method of any preceding claim, wherein the plant cell is in a plant.

24. The method of claim 23, wherein the plant is selected from: cotton, cantaloupe, radicchio, papaya, plum, peanut, oilseed rape, canola, sunflower, safflower, olive, sesame, hazelnut, almond, avocado, bay, pumpkin/squash, linseed, soya, pistachio, borage, maize, wheat, rye, oats, sorghum and millet, triticale, rice, barley, cassava, potato, sugarbeet, egg plant, alfalfa, perennial grasses, forage plants, oil palm, vegetables (brassicas, root vegetables, tuber vegetables, pod vegetables, fruiting vegetables, onion vegetables, leafy vegetables and stem vegetable), buckwheat, Jerusalem artichoke, broad bean, vetches, lentil, dwarf bean, lupin, clover, lucerne, tobacco, tomato, ornamental plants and marijuana.

25. The method of claim 23 or 24, wherein the method further comprises the step of:
(ii) growing the plant to seed.

26. The method of claim 25, wherein the method further comprises the step of:
(iii) growing the seed(s) obtained in step (ii).

27. The method of claim 26, wherein the method further comprises repeating steps (ii) and (iii).

28. A method for identifying and/or selecting a plant cell with a trait of interest, the method comprising:
(i) reducing or abolishing the expression of at least one TONSOKU nucleic acid sequence and/or reducing or abolishing the level of a TONSOKU polypeptide and/or reducing or abolishing an activity of a TONSOKU polypeptide in the plant cell;
(ii) selecting at least one plant cell with a trait of interest; and optionally
(iii) genotyping the plant cell obtained in step (ii).

29. The method of claim 28, wherein the method further comprises growing the plant cell obtained in step (i).

30. The method of claim 29, wherein the method further comprises growing the plant cell obtained in step (i) into a plant.

31. The method of claim 30, wherein the method further comprises growing the plant to seed to obtain progeny of the plant.

32. The method as claimed in claims 28 to 31, wherein selecting at least one plant cell with a trait of interest is determined by:
(i) inspecting morphological features of the at least one plant cell;
(ii) genotyping the at least one plant cell;
(iii) transcriptomic analysis of the at least one plant cell;
(iv) metabolomic analysis of the at least one plant cell; or
(v) assessing the behaviour of the at least one plant cell in a phenotypic assay.

33. A method for screening a population of plant cells and identifying and/or selecting a plant cell with a trait of interest, the method comprising:
(i) reducing or abolishing the expression of at least one TONSOKU nucleic acid sequence and/or reducing or abolishing the level of a TONSOKU polypeptide and/or reducing or abolishing an activity of a TONSOKU polypeptide in the plant cell;
(ii) selecting at least one plant cell with a trait of interest; and optionally
(iii) genotyping the plant cell obtained in step (ii).

34. The method of claim 33, wherein the method further comprises growing the plant cells obtained in step (i) to form a population of plant cells.

35. The method of claim 33 or claim 34, wherein the method further comprises screening the population of plant cells obtained in step (i) for reduced expression of at least one TONSOKU nucleic acid sequence or a reduced level of a TONSOKU polypeptide or reduced activity of a TONSOKU polypeptide in the plant cell prior to step (ii) and (iii).

36. The method as claimed in claims 27 to 33, wherein the trait of interest is selected from: insect resistance, disease resistance, herbicide tolerance, male sterility, abiotic stress tolerance, altered phosphorus utilisation, altered antioxidants, altered fatty acids, altered essential amino acids, altered carbohydrates, sequences involved in site-specific recombination, altered development, or altered morphology (such as size and pigmentation).

37. A population of plant cells, plant parts or plants obtained by the methods of any preceding claim.


SEQUENCE LISTING (12 sequences)

SEQ ID NO: 1 - protein, 1311 aa, Arabidopsis thaliana
MGRLDVAAAKRAYRKAEEVGDRREQARWANNVGDILKNHGEYVDALKWFRIDYDISVKYLPGKDLLPTCQSLGEIYLRLENFEEALIYQKKHLQLAEEANDTVEKQRACTQLGRTYHEMFLKSEDDCEAIQSAKKYFKKAMELAQILKEKPPPGESSGFLEEYINAHNNIGMLDLDLDNPEAARTILKKGLQICDEEEVREYDAARSRLHHNLGNVFMALRSWDEAKKHIEMDINICHKINHVQGEAKGYINLAELHNKTQKYIDALLCYGKASSLAKSMQDESALVEQIEHNTKIVKKSMKVMEELREEELMLKKLSAEMTDAKGTSEERKSMLQVNACLGSLIDKSSMVFAWLKHLQYSKRKKKISDELCDKEKLSDAFMIVGESYQNLRNFRKSLKWFIRSYEGHEAIGNLEGQALAKINIGNGLDCIGEWTGALQAYEEGYRIALKANLPSIQLSALEDIHYIHMMRFGNAQKASELKETIQNLKESEHAEKAECSTQDECSETDSEGHANVSNDRPNACSSPQTPNSLRSERLADLDEANDDVPLISFLQPGKRLFKRKQVSGKQDADTDQTKKDFSVVADSQQTVAGRKRIRVILSDDESETEYELGCPKDSSHKVLRQNEEVSEESMYFDGAINYTDNRAIQDNVEEGSCSYTPLHPIKVAPNVSNCRSLSNNIAVETTGRRKKGSQCDVGDSNGTSCKTGAALVNFHAYSKTEDRKIKIEIENEHIALDSCSHDDESVKVELTCLYYLQLPDDEKSKGLLPIIHHLEYGGRVLKPLELYAILRDSSENVVIEASVDGWVHKRLMKLYMDCCQSLSEKPSMKLLKKLYISEVEDDINVSECELQDISAAPLLCALHVHNIAMLDLSHNMLGNGTMEKLKQLFASSSQMYGALTLDLHCNRFGPTALFQICECPVLFTRLEVLNVSRNRLTDACGSYLSTIVKNCRALYSLNVEHCSLTSRTIQKVANALDSKSGLSQLCIGYNNPVSGSSIQNLLAKLATLSSFAELSMNGIKLSSQVVDSLYALVKTPSLSKLLVGSSGIGTDGAIKVTESLCYQKEETVKLDLSCCGLASSFFIKLNQDVTLTSSILEFNVGGNPITEEGISALGELLRNPCSNIKVLILSKCHLKLAGLLCIIQALSDNKNLEELNLSDNAKIEDETVFGQPVKERSVMVEQEHGTCKSVTSMDKEQELCETNMECDDLEVADSEDEQIEEGTATSSSLSLPRKNHIVKELSTALSMANQLKILDLSNNGFSVEALETLYMSWSSSSSRTGIAQRHVKEETVHFYVEGKMCCGVKSCCRKD

SEQ ID NO: 2 - DNA, 1500 bp, Arabidopsis thaliana
cctggaaaaccgatgtcacagtcgatcatctcatccattcgcaactgaatcagaactcaagaagtcatcataacgaagcaaagccacagaaacaagaggagactgtttttcatgatacttgtgagttggttagtcactcgtgtaactcagattgcccacgatcagatgaggaagataagcaatgcgtcgatgccaccaaaggagaagacaagagctccattcaagaagtagaagaagcaaccgaaccagtaagtttggaggaagaagaaaggttaagacaagagctggaggagatagaagctaagtatcaggaagatatgaaagagatagcaacgaaaagagaagaggccattatggagacgaagaaaaagttgtctctgatgaagttaaagtaatagccaaaaaagctcaaagaaaacgttgatactgatgaagagcttttgtgtttttaatctcttttgtttaatttgttggttggaggagaagtgtagaaagatgaagggtttctatttgattaattgagatttaatttggttggttgttacaagttagaacataaaaaatggttcctgttaaaatgttctaagagattgtccattatatatgattttgtataaattgaacatgtaattagttaatagccaactattgtaataaaagtaatcaagccttttcgtgtaaggaatcaatcaacagagacgaaaatgtagtaattaattataaccattaagaggaagtcgggaaaccaaagaaatctaacattaagtctttgaagaacacaaagcataatcaagcatagagaacaacatggcaaaatcatcaaaatcagaatcactgatctccaggaagtgtcttgatgatgtcggaatcaccaggatcaacgatgctgaggcaagaaactcggaagtatttaccacaagcagtacccaaatcaacattgttgccattgtagcgatgaactccaactttagcaagcatcgcatagtattcaatctctgaccttctcaacggtgggcaattgctagatatcaatatcagcttacctaaaccccccaacaatatccaacaattattcaactaaattacgaggaagacgaacactataatcaatcgatgaagagggattttaaatttttacctttggagctgcgaagggatttgagaacagacttgtatccaagagtgtactttccactcttcatcacaagagctaatctgctgttgattccttcatgggacttcttcgccttcttctccgcaaccatttttcaccgccgggaagattcagatcgcaggtttacaagagagagttcttcttcgggttcgggcggcgcaaaatgatagtttatatagcgagtgccttagaacccttagggtttttttgttttcttgtcaggagacaggaggatataagaagcccaaaataaactcgacccaaggcccaaactaaaaggcctataacttcaggatttagggtatgaaaatttctaatttaccctt

SEQ ID NO: 3 - DNA, 7350 bp, Arabidopsis thaliana
gaattttggcgggatagtttgggatgggaccaaaaatttggcgactggagaaaatgagaaaatcaaaatcactgagaaagaaatttcgagaaatctgaaaatcggaaggaagaaaacaaaaacctttcaattgaagaacggagaaatcatcatccgatgggtcgattagatgtagctgcggcgaagagagcgtaccggaaagcagaagaagtgggtgaccggagagaacaggcgaggtgggctaacaatgtcggcgatatccttaagaatcatggagagtacgttgatgctctcaagtggtttaggattgattacgatatctccgtcaagtatttacctgggaaagatttgttacctacttgtcagtctcttggcgagatctatctccgcctcgaaaatttcgaagaagccttgatttatcaggtaagccctcttgaatcaattgctttttcctacttggttattgttggcttcctgaattttccgtgaataattttggtgtttgagtttttcattttgaatttgtgtttttttctggtggttgcagaagaagcatttacagctagctgaagaagctaatgacactgtggagaagcaaagagcatgtactcaacttggacgtacttaccatgaaatgttcttgaagtctgaggatgattgtgaagccattcagagtgctaaaaagtactttaagaaagccatggaacttgcacagattctcaaggagaaaccacctcctggagaatctagcggattccttgaggagtatattaacgcacataacaacatcggtatgcttgaccttgatcttgataatcctgaagcagcccgtactattcttaagaaagggctgcagatttgcgatgaagaggaggtgagagagtatgatgctgctcggagtaggcttcatcataaccttggaaacgtttttatggcgctgagaagttgggatgaagcaaagaaacacattgagatggatattaatatctgtcataagattaatcatgtccaaggagaagcgaaggggtatatcaatctcgctgaattacacaacaagacccaaaagtacattgatgctcttttatgttatggtaaagcttctagtctagcgaaatctatgcaagacgagagtgcattggttgaacagatagagcataataccaagatagtcaagaaatccatgaaagttatggaagaattgagagaagaagagcttatgcttaagaagttgtctgcagaaatgactgatgccaaaggcacttcggaggaacgaaagtctatgctccaagtaaatgcttgtcttggaagtcttattgataaatctagcatggtattcgcatggctgaaggtgagttttataacttaaacactccttcctttttagtcctatcactccaccccatgttcgcatttatttgaaaagtttccagaagttaaagttgtccatcgtaggggtttttaatgatgaataagcattgtgagatttcatcaggtagtatggagtaggaaaaatatgctattttcttagatttgatttaagttttgtgaacttctgctattgacactgtcttttcagatcagtcaggaagactatattatcaaagaattacatgattcttgttctctcaagaaaacctatcttttgaatgctgggataatatctttgttctgaacttgcaaagtaaagttattatgtggcaaaacgatgattattctgtatcatacggatactgagtgatccaagtctctgcatcactgtttcaatgacttgtgatatagttttgaaagttaagtaggaggctgccatttgaagtttgcatgcaactaaagggttgctatttcttctttgaatgtcttagcatcttcaatattcaaaaaggaagaagaaaatatcagatgaactctgtgacaaggaaaagctgagtgatgccttcatgattgttggagaatcttaccaaaatctcagaaatttcagaaagtccctgaagtggttcataagaagttatgagggacatgaagcaattggtaatctggaggtgagatttgtttgcttgcacgattaattataaaaacctatgttcactactgtcatcagaatttgattcacaaaaccagaaataattcattaggcctctactgaacattttctgtggaaaactgattataccttttcttggatttgtcaatattatagctattcttctttcctgattctaatattcacttatggtggtctcttgtagggtcaagcactagcgaagattaatattggtaatggtttggactgtattggggaatggacaggagcacttcaggcatatgaagaggggtacaggtagatccaattataagtaatctttatcaaactgcgcatttgagctattatttggttatgtttgtgattcagtcctagtaaatctacttattaattttccttgagagaactgataattccattgaacaatatgacggcgatgaaactcatttttttcttaaaatggaaagaacacttgaagcagagcaaatgtgaatgtgctataaagtacttaactgcttgttggttgtccctttcgactaagttcacgaattactgcactatggcttctgaataaataatacaatgtactttgaatcagtacttctcatgatagtggataattatagcacattttgcattttcaatcacttaaaatattttttctgtgactttcttctgctatattcaaacacatcgcatatacatttacgtgaatttatacacacatactgcatgctaataaattaactattggtctttctggatttattttcatttgatcctgcagaattgctttgaaagctaatcttccttcaatccagctttctgcactggaagatatacactatatccatatgatgagatttgggaatgctcaaaaagccaggtaacaattactgttttgtcactggacggaatatggatagacaccaaatctggtgtaaggtttgcagtttcaagtatttcattttactcatatattatttctactgtctagtgaattgaaggaaacaatacaaaatctgaaggagtcagaacatgctgagaaagccgaatgtagtacacaagatgaatgctctgaaactgactcagaagggcatgcgaatgtatcgaatgataggccaaatgcatgtagctcaccgcaaacaccaaattcacttagatcagaacggttagcagatctggatgaagcaaatgatgatgtgccactaatttcatttctccagcctggaaaacgtctgttcaaaaggaaacaagtttcaggaaaacaagatgctgacactgatcagacgaagaaagatttctctgtagtagcagactctcagcagacagttgctggtcgaaagcgtattcgagtaatcctctctgatgatgaaagtgagaccgaatatgagctgggatgccctaaagacagttctcacaaagttctaaggcagaatgaagaggtttctgaggaaagtatgtattttgatggtgctattaattatacggataatcgtgccatccaagataatgtagaagaaggttcttgctcgtatacgcctctccatcctattaaggtggctccaaatgtcagcaattgtagatctttgagtaataatatagctgttgaaacaactggtcgtcgtaaaaaaggatctcaatgtgatgttggcgactccaacggcacgtcctgcaaaactggagctgctctcgtgaacttccacgcttactcaaaaactgaggatgtgagcaactgtgatctggtttttgagttatcattgaccattcttgggattggatttcatttatttttctacttcgtccaatcttcttcatgataactatatgttttacttgttgcagcgaaaaataaaaattgaaattgaaaatgaacacatagctttagactcctgttctcacgatgatgagtctgtgaaggtggaacttacttgcctatactatttacagcttcctgacgatgagaaatctaaaggtatgtgcttttgttttcttagcaaaactttaggatgatcccagttcggatcagtctctataatgcatgatcccagttcggatcagtcctataattctcatctcacgcttaataacatttcttttgctttttgatatcattccccttgtttcctagcacgttttaagttttgctctaaaagtttgaatctttgaacattcaatttgcgttaggtctgttgccgatcattcatcatttggaatatggtggaagagttctgaaaccattggaactatatgcgattctcagggactcttctgaaaatgttgttattgaagcttccgttgatggtaagtatttccttgatagaattggaatctactcatgatatttggatgtatgattgtcaagctgatcattctataaatttgttttcatcacaaattgttctctcactttttacatgattgtgctgaaccgctgtattggcttttaagattatggtcattgattcttccctcttatttatacaccacggctgaatcagcatgaaattaatttgttttcaggctgggttcacaagcgcctgatgaaactatacatggactgttgccagtcgttgtcagagaaacccagtatgaaattgcttaagaaattatatatttcggaggtgagagtattaccccaaattttagcggttaatgtatgaaatattttcttctctttgtttgcttttcaacctacttaaagctagctagttacaaattcttactttatttgatgtataatctgaatggttatttcgttgtatgtttatcaggtagaagatgatatcaatgtgtcagaatgtgaactgcaagacatatcagctgctccattattgtgtgccctccatgtccacaatattgctatgttggatctctcccacaatatgctaggtgaaagttgcctctgacgtcttacttaatttaatgagctgacctaagtgagttagttggttatgcatagggaactactaggaaattcagaagtgttaatttccatcgtctcattggttgttagggaatggaacaatggagaaattgaaacaactttttgcctcatcaagccagatgtatggtgctttaactttggatttgcactgcaatcgatttggtccaactgctttgtttcaggtacactactaggcccaaagctagaaaatttcacatattcatgttattttcgtattatttaatatactcctctttaccagatctgtgaatgccctgttctgttcactcgacttgaagtcctcaatgtgtccaggaatcgacttacagatgcttgtggatcatacctctcaactatagtgaaaaattgccggggtatagatttttttttttttttttttaaattatgataattcatttacagtatctaaatgccctgatggtatgttttgtttcttggtttcactggtgtcttataaacccagtagatagatatatgaaatacctgatattaggtttaataatcttaaacattttcttccattcactagcttacattaatgtgtccccttttgtttcttagcactttacagcttgaatgtggaacattgttcacttacatcaagaacaatccaaaaggtagctaatgctttggattcgaagtcaggactttcacaactctgtataggtgatctttctaatttgttatgtacattcaatttattttttttatctcgtttcagtttgctgaagttggtggatccgtatatggcaggttataataatcctgtttcagggagtagtattcaaaacctcttggctaaattggctactctaagcaggttgaaagaaacacattttaaagctgttttttttttatacgtaaatccatctaacatgatcatatgtcaaaacactgcagctttgcagaactgagcatgaatggcataaagctgagcagccaagttgttgatagcctttatgcacttgttaagactccatctctgtcaaaacttttggttggcagcagtggaataggaacggtaatgatatgtttagcattcaaaattgaattcttatattgtgataaatacatctttttttatctgacgatactatacaaattattctaggacggggctataaaagttactgaatctctatgttatcagaaggaagaaactgtgaagctcgacctttcatgttgtggactagcttcctctttctttattaagctcaaccaagatgttactctaacctctagcattcttgagtttaatgttggaggaaatccaatcaccgaagaggtatgttttctatgactcaacatcctaaagctcttttatctaactctgttgaggctgcaatggtgatagaataagctaaagaatttgcaatcattcaacatgtgattttaagttcatgtcttctcaaagcataactgactctctgaaacactaaacaaacagggaatcagtgcacttggggagctgcttaggaatccttgttcaaacataaaagttcttattctaagcaagtgtcatctgaagctcgctgggcttctatgcataattcaagcactttcaggtctgaagtattcttgtagctgctattaaacaaaagatcttctcctttttaaactatcaactaaatgctctgcagataataagaatcttgaagagcttaatctttctgacaatgctaagatagaagatgagactgtgtttggccaacctgtgaaggaaagatcagtaatggtagagcaagaacatggaacatgtaaatctgtcacctcaatggacaaagaacaagagctatgtgaaaccaatatggagtgtgatgatctcgaagttgcagacagcgaagatgaacaaatagaggaaggaactgcaacctcgagtagtcttagtttgccacgcaagaaccatatcgtgaaagagctttctaccgctctttcaatggctaaccagttgaagattctggacttaagcaacaatgggttctcagttgaagccttggaaacattatacatgtcatggtcatcatcaagctcccgaactggcatcgcccaaaggcatgtaaaagaagagactgtccatttttatgtcgaaggaaagatgtgttgcggagtcaaatcatgctgcagaaaggactgaagaagatcttgtctgaaactgtatttgccaataataaacctctgtttttaaatattgagtatttttatttagagcgtttgcagaaatttttacatattgatatttacacatttgggttgtgatgtgtaaatttgctgcagtttaagcgttaatgctcatataaatttagtgacgttaatcttatgcaactttttaaaaaatgtaaaaatt

SEQ ID NO: 4 - DNA, 4302 bp, Arabidopsis thaliana
gaattttggcgggatagtttgggatgggaccaaaaatttggcgactggagaaaatgagaaaatcaaaatcactgagaaagaaatttcgagaaatctgaaaatcggaaggaagaaaacaaaaacctttcaattgaagaacggagaaatcatcatccgatgggtcgattagatgtagctgcggcgaagagagcgtaccggaaagcagaagaagtgggtgaccggagagaacaggcgaggtgggctaacaatgtcggcgatatccttaagaatcatggagagtacgttgatgctctcaagtggtttaggattgattacgatatctccgtcaagtatttacctgggaaagatttgttacctacttgtcagtctcttggcgagatctatctccgcctcgaaaatttcgaagaagccttgatttatcagaagaagcatttacagctagctgaagaagctaatgacactgtggagaagcaaagagcatgtactcaacttggacgtacttaccatgaaatgttcttgaagtctgaggatgattgtgaagccattcagagtgctaaaaagtactttaagaaagccatggaacttgcacagattctcaaggagaaaccacctcctggagaatctagcggattccttgaggagtatattaacgcacataacaacatcggtatgcttgaccttgatcttgataatcctgaagcagcccgtactattcttaagaaagggctgcagatttgcgatgaagaggaggtgagagagtatgatgctgctcggagtaggcttcatcataaccttggaaacgtttttatggcgctgagaagttgggatgaagcaaagaaacacattgagatggatattaatatctgtcataagattaatcatgtccaaggagaagcgaaggggtatatcaatctcgctgaattacacaacaagacccaaaagtacattgatgctcttttatgttatggtaaagcttctagtctagcgaaatctatgcaagacgagagtgcattggttgaacagatagagcataataccaagatagtcaagaaatccatgaaagttatggaagaattgagagaagaagagcttatgcttaagaagttgtctgcagaaatgactgatgccaaaggcacttcggaggaacgaaagtctatgctccaagtaaatgcttgtcttggaagtcttattgataaatctagcatggtattcgcatggctgaagcatcttcaatattcaaaaaggaagaagaaaatatcagatgaactctgtgacaaggaaaagctgagtgatgccttcatgattgttggagaatcttaccaaaatctcagaaatttcagaaagtccctgaagtggttcataagaagttatgagggacatgaagcaattggtaatctggagggtcaagcactagcgaagattaatattggtaatggtttggactgtattggggaatggacaggagcacttcaggcatatgaagaggggtacagaattgctttgaaagctaatcttccttcaatccagctttctgcactggaagatatacactatatccatatgatgagatttgggaatgctcaaaaagccagtgaattgaaggaaacaatacaaaatctgaaggagtcagaacatgctgagaaagccgaatgtagtacacaagatgaatgctctgaaactgactcagaagggcatgcgaatgtatcgaatgataggccaaatgcatgtagctcaccgcaaacaccaaattcacttagatcagaacggttagcagatctggatgaagcaaatgatgatgtgccactaatttcatttctccagcctggaaaacgtctgttcaaaaggaaacaagtttcaggaaaacaagatgctgacactgatcagacgaagaaagatttctctgtagtagcagactctcagcagacagttgctggtcgaaagcgtattcgagtaatcctctctgatgatgaaagtgagaccgaatatgagctgggatgccctaaagacagttctcacaaagttctaaggcagaatgaagaggtttctgaggaaagtatgtattttgatggtgctattaattatacggataatcgtgccatccaagataatgtagaagaaggttcttgctcgtatacgcctctccatcctattaaggtggctccaaatgtcagcaattgtagatctttgagtaataatatagctgttgaaacaactggtcgtcgtaaaaaaggatctcaatgtgatgttggcgactccaacggcacgtcctgcaaaactggagctgctctcgtgaacttccacgcttactcaaaaactgaggatcgaaaaataaaaattgaaattgaaaatgaacacatagctttagactcctgttctcacgatgatgagtctgtgaaggtggaacttacttgcctatactatttacagcttcctgacgatgagaaatctaaaggtctgttgccgatcattcatcatttggaatatggtggaagagttctgaaaccattggaactatatgcgattctcagggactcttctgaaaatgttgttattgaagcttccgttgatggctgggttcacaagcgcctgatgaaactatacatggactgttgccagtcgttgtcagagaaacccagtatgaaattgcttaagaaattatatatttcggaggtagaagatgatatcaatgtgtcagaatgtgaactgcaagacatatcagctgctccattattgtgtgccctccatgtccacaatattgctatgttggatctctcccacaatatgctagggaatggaacaatggagaaattgaaacaactttttgcctcatcaagccagatgtatggtgctttaactttggatttgcactgcaatcgatttggtccaactgctttgtttcagatctgtgaatgccctgttctgttcactcgacttgaagtcctcaatgtgtccaggaatcgacttacagatgcttgtggatcatacctctcaactatagtgaaaaattgccgggcactttacagcttgaatgtggaacattgttcacttacatcaagaacaatccaaaaggtagctaatgctttggattcgaagtcaggactttcacaactctgtataggttataataatcctgtttcagggagtagtattcaaaacctcttggctaaattggctactctaagcagctttgcagaactgagcatgaatggcataaagctgagcagccaagttgttgatagcctttatgcacttgttaagactccatctctgtcaaaacttttggttggcagcagtggaataggaacggacggggctataaaagttactgaatctctatgttatcagaaggaagaaactgtgaagctcgacctttcatgttgtggactagcttcctctttctttattaagctcaaccaagatgttactctaacctctagcattcttgagtttaatgttggaggaaatccaatcaccgaagagggaatcagtgcacttggggagctgcttaggaatccttgttcaaacataaaagttcttattctaagcaagtgtcatctgaagctcgctgggcttctatgcataattcaagcactttcagataataagaatcttgaagagcttaatctttctgacaatgctaagatagaagatgagactgtgtttggccaacctgtgaaggaaagatcagtaatggtagagcaagaacatggaacatgtaaatctgtcacctcaatggacaaagaacaagagctatgtgaaaccaatatggagtgtgatgatctcgaagttgcagacagcgaagatgaacaaatagaggaaggaactgcaacctcgagtagtcttagtttgccacgcaagaaccatatcgtgaaagagctttctaccgctctttcaatggctaaccagttgaagattctggacttaagcaacaatgggttctcagttgaagccttggaaacattatacatgtcatggtcatcatcaagctcccgaactggcatcgcccaaaggcatgtaaaagaagagactgtccatttttatgtcgaaggaaagatgtgttgcggagtcaaatcatgctgcagaaaggactgaagaagatcttgtctgaaactgtatttgccaataataaacctctgtttttaaatattgagtatttttatttagagcgtttgcagaaatttttacatattgatatttacacatttgggttgtgatgtgtaaatttgctgcagtttaagcgttaatgctcatataaatttagtgacgttaatcttatgcaactttttaaaaaatgtaaaaatt

SEQ ID NO: 5 - DNA, 5 bp, Artificial Sequence
  A single unit sequence
attcg

SEQ ID NO: 6 - DNA, 10 bp, Artificial Sequence
  A polynucleotide with two tandem repeats of the unit sequence
attcgattcg

SEQ ID NO: 7 - DNA, 15 bp, Artificial Sequence
  A polynucleotide with three tandem repeats of the unit sequence
attcgattcgattcg

SEQ ID NO: 8 - DNA, 20 bp, Artificial Sequence
  A polynucleotide with four tandem repeats of the unit sequence
attcgattcgattcgattcg

SEQ ID NO: 9 - DNA, 7 bp, Artificial Sequence
  A single unit sequence
tatacag

SEQ ID NO: 10 - DNA, 43 bp, Artificial Sequence
  WT sequence from figure 2
  misc_feature (1)..(17): n is a, c, g, or t
  misc_feature (18)..(18): unit sequence
  misc_feature (19)..(35): n is a, c, g, or t
  misc_feature (36)..(36): unit sequence
  misc_feature (37)..(43): n is a, c, g, or t
nnnnnnnnnnnnnnnnnnnnnnnnnnnnnnnnnnnnnnnnnnn

SEQ ID NO: 11 - DNA, 44 bp, Artificial Sequence
  Sequence A from figure 2
  misc_feature (1)..(17): n is a, c, g, or t
  misc_feature (18)..(18): unit sequence
  misc_feature (19)..(19): unit sequence
  misc_feature (20)..(36): n is a, c, g, or t
  misc_feature (37)..(37): unit sequence
  misc_feature (38)..(44): n is a, c, g, or t
nnnnnnnnnnnnnnnnnnnnnnnnnnnnnnnnnnnnnnnnnnnn

SEQ ID NO: 12 - DNA, 46 bp, Artificial Sequence
  Sequence B from Figure 2
  misc_feature (1)..(17): n is a, c, g, or t
  misc_feature (18)..(18): unit sequence
  misc_feature (19)..(19): unit sequence
  misc_feature (20)..(36): n is a, c, g, or t
  misc_feature (37)..(37): unit sequence
  misc_feature (38)..(38): unit sequence
  misc_feature (39)..(39): unit sequence
  misc_feature (40)..(46): n is a, c, g, or t
nnnnnnnnnnnnnnnnnnnnnnnnnnnnnnnnnnnnnnnnnnnnnn